SEQUENCE LISTING (9 sequences)

SEQ ID NO: 1 - DNA, 7536 bp, Homo sapiens
atggaggaggtggtgattgccggcatgtccgggaagctgccagagtcggagaacttgcaggagttctgggacaacctcatcggcggtgtggacatggtcacggacgatgaccgtcgctggaaggcggggctctacggcctgccccggcggtccggcaagctgaaggacctgtctaggtttgatgcctccttcttcggagtccaccccaagcaggcacacacgatggaccctcagctgcggctgctgctggaagtcacctatgaagccatcgtggacggaggcatcaacccagattcactccgaggaacacacactggcgtctgggtgggcgtgagcggctctgagacctcggaggccctgagccgagaccccgagacactcgtgggctacagcatggtgggctgccagcgagcgatgatggccaaccggctctccttcttcttcgacttcagagggcccagcatcgcactggacacagcctgctcctccagcctgatggccctgcagaacgcctaccaggccatccacagcgggcagtgccctgccgccatcgtggggggcatcaatgtcctgctgaagcccaacacctccgtgcagttcttgaggctggggatgctcagccccgagggcacctgcaaggccttcgacacagcggggaatgggtactgccgctcggagggtgtggtggccgtcctgctgaccaagaagtccctggcccggcgggtgtacgccaccatcctgaacgccggcaccaatacagatggcttcaaggagcaaggcgtgaccttcccctcaggggatatccaggagcagctcatccgctcgttgtaccagtcggccggagtggcccctgagtcatttgaatacatcgaagcccacggcacaggcaccaaggtgggcgacccccaggagctgaatggcatcacccgagccctgtgcgccacccgccaggagccgctgctcatcggctccaccaagtccaacatggggcacccggagccagcctcggggctggcagccctggccaaggtgctgctgtccctggagcacgggctctgggcccccaacctgcacttccatagccccaaccctgagatcccagcgctgttggatgggcggctgcaggtggtggaccagcccctgcccgtccgtggcggcaacgtgggcatcaactcctttggcttcgggggctccaacgtgcacatcatcctgaggcccaacacgcagccgccccccgcacccgccccacatgccaccctgccccgtctgctgcgggccagcggacgcacccctgaggccgtgcagaagctgctggagcagggcctccggcacagccaggacctggctttcctgagcatgctgaacgacatcgcggctgtccccgccaccgccatgcccttccgtggctacgctgtgctgggtggtgagcgcggtggcccagaggtgcagcaggtgcccgctggcgagcgcccgctctggttcatctgctctgggatgggcacacagtggcgcgggatggggctgagcctcatgcgcctggaccgcttccgagattccatcctacgctccgatgaggctgtgaagccattcggcctgaaggtgtcacagctgctgctgagcacagacgagagcacctttgatgacatcgtccattcgtttgtgagcctgactgccatccagataggcctcatagacctgctgagctgcatggggctgaggccagatggcatcgtcggccactccctgggggaggtggcctgtggctacgccgacggctgcctgtcccaggaggaggccgtcctcgctgcctactggaggggacagtgcatcaaagaagcccatctcccgccgggcgccatggcagccgtgggcttgtcctgggaggagtgtaaacagcgctgccccccgggcgtggtgcccgcctgccacaactccaaggacacagtcaccatctcgggacctcaggccccggtgtttgagttcgtggagcagctgaggaaggagggtgtgtttgccaaggaggtgcggaccggcggtatggccttccactcctacttcatggaggccatcgcacccccactgctgcaggagctcaagaaggtgatccgggagccgaagccacgttcagcccgctggctcagcacctctatccccgaggcccagtggcacagcagcctggcacgcacgtcctccgccgagtacaatgtcaacaacctggtgagccctgtgctgttccaggaggccctgtggcacgtgcctgagcacgcggtggtgctggagatcgcgccccacgccctgctgcaggctgtcctgaagcgtggcctgaagccgagctgcaccatcatccccctgatgaagaaggatcacagggacaacctggagttcttcctggccggcatcggcaggctgcacctctcaggcatcgacgccaaccccaatgccttgttcccacctgtggagttcccagctccccgaggaactcccctcatctccccactcatcaagtgggaccacagcctggcctgggacgtgccggccgccgaggacttccccaacggttcaggttccccctcagccgccatctacaacatcgacaccagctccgagtctcctgaccactacctggtggaccacaccctcgacggtcgcgtcctcttccccgccactggctacctgagcatagtgtggaagacgctggcccgcgccctgggcctgggcgtcgagcagctgcctgtggtgtttgaggatgtggtgctgcaccaggccaccatcctgcccaagactgggacagtgtccctggaggtacggctcctggaggcctcccgtgccttcgaggtgtcagagaacggcaacctggtagtgagtgggaaggtgtaccagtgggatgaccctgaccccaggctcttcgaccacccggaaagccccacccccaaccccacggagcccctcttcctggcccaggctgaagtttacaaggagctgcgtctgcgtggctacgactacggccctcatttccagggcatcctggaggccagcctggaaggtgactcggggaggctgctgtggaaggataactgggtgagcttcatggacaccatgctgcagatgtccatcctgggctcggccaagcacggcctgtacctgcccacccgtgtcaccgccatccacatcgaccctgccacccacaggcagaagctgtacacactgcaggacaaggcccaagtggctgacgtggtggtgagcaggtggctgagggtcacagtggccggaggcgtccacatctccgggctccacactgagtcggccccgcggcggcagcaggagcagcaggtgcccatcctggagaagttttgcttcactccccacacggaggaggggtgcctgtctgagcgcgctgccctgcaggaggagctgcaactgtgcaaggggctggtgcaggcactgcagaccaaggtgacccagcaggggctgaagatggtggtgcccggactggatggggcccagatcccccgggacccctcacagcaggaactgccccggctgttgtcggctgcctgcaggcttcagctcaacgggaacctgcagctggagctggcgcaggtgctggcccaggagaggcccaagctgccagaggaccctctgctcagcggcctcctggactccccggcactcaaggcctgcctggacactgccgtggagaacatgcccagcctgaagatgaaggtggtggaggtgctggctggccacggtcacctgtattcccgcatcccaggcctgctcagcccccatcccctgctgcagctgagctacacggccaccgaccgccacccccaggccctggaggctgcccaggccgagctgcagcagcacgacgttgcccagggccagtgggatcccgcagaccctgcccccagcgccctgggcagcgccgacctcctggtgtgcaactgtgctgtggctgccctcggggacccggcctcagctctcagcaacatggtggctgccctgagagaagggggctttctgctcctgcacacactgctccgggggcaccccctcggggacatcgtggccttcctcacctccactgagccgcagtatggccagggcatcctgagccaggacgcgtgggagagcctcttctccagggtgtcgctgcgcctggtgggcctgaagaagtccttctacggctccacgctcttcctgtgccgccggcccaccccgcaggacagccccatcttcctgccggtggacgataccagcttccgctgggtggagtctctgaagggcatcctggctgacgaagactcttcccggcctgtgtggctgaaggccatcaactgtgccacctcgggcgtggtgggcttggtgaactgtctccgccgagagcccggcgggaaccgcctccggtgtgtgctgctctccaacctcagcagcacctcccacgtcccggaggtggacccgggctccgcagaactgcagaaggtgttgcagggagacctggtgatgaacgtctaccgcgacggggcctggggggctttccgccacttcctgctggaggaggacaagcctgaggagccgacggcacatgcctttgtgagcaccctcacccggggggacctgtcctccatccgctgggtctgctcctcgctgcgccatgcccagcccacctgccctggcgcccagctctgcacggtctactacgcctccctcaacttccgcgacatcatgctggccactggcaagctgtcccctgatgccatcccagggaagtggacctcccaggacagcctgctaggtatggagttctcgggccgagacgccagcggcaagcgtgtgatgggactggtgcctgccaagggcctggccacctctgtcctgctgtcaccggacttcctctgggatgtgccttccaactggacgctggaggaggcggcctcggtgcctgtcgtctacagcacggcctactacgcgctggtggtgcgtgggcgggtgcgccccggggagacgctgctcatccactcgggctcgggcggcgtgggccaggccgccatcgccatcgccctcagtctgggctgccgcgtcttcaccaccgtggggtcggctgagaagcgggcgtacctccaggccaggttcccccagctcgacagcaccagcttcgccaactcccgggacacatccttcgagcagcatgtgctgtggcacacgggcgggaagggcgttgacctggtcttgaactccttggcggaagagaagctgcaggccagcgtgaggtgcttggctacgcacggtcgcttcctggaaattggcaaattcgacctttctcagaaccacccgctcggcatggctatcttcctgaagaacgtgacattccacggggtcctactggatgcgttcttcaacgagagcagtgctgactggcgggaggtgtgggcgcttgtgcaggccggcatccgggatggggtggtacggcccctcaagtgcacggtgttccatggggcccaggtggaggacgccttccgctacatggcccaagggaagcacattggcaaagtcgtcgtgcaggtgcttgcggaggagccggaggcagtgctgaagggggccaaacccaagctgatgtcggccatctccaagaccttctgcccggcccacaagagctacatcatcgctggtggtctgggtggcttcggcctggagttggcgcagtggctgatacagcgtggggtgcagaagctcgtgttgacttctcgctccgggatccggacaggctaccaggccaagcaggtccgccggtggaggcgccagggcgtacaggtgcaggtgtccaccagcaacatcagctcactggagggggcccggggcctcattgccgaggcggcgcagcttgggcccgtgggcggcgtcttcaacctggccgtggtcttgagagatggcttgctggagaaccagaccccagagttcttccaggacgtctgcaagcccaagtacagcggcaccctgaacctggacagggtgacccgagaggcgtgccctgagctggactactttgtggtcttctcctctgtgagctgcgggcgtggcaatgcgggacagagcaactacggctttgccaattccgccatggagcgtatctgtgagaaacgccggcacgaaggcctcccaggcctggccgtgcagtggggcgccatcggcgacgtgggcattttggtggagacgatgagcaccaacgacacgatcgtcagtggcacgctgccccagcgcatggcgtcctgcctggaggtgctggacctcttcctgaaccagccccacatggtcctgagcagctttgtgctggctgagaaggctgcggcctatagggacagggacagccagcgggacctggtggaggccgtggcacacatcctgggcatccgcgacttggctgctgtcaacctggacagctcactggcggacctgggcctggactcgctcatgagcgtggaggtgcgccagacgctggagcgtgagctcaacctggtgctgtccgtgcgcgaggtgcggcaactcacgctccggaaactgcaggagctgtcctcaaaggcggatgaggccagcgagctggcatgccccacgcccaaggaggatggtctggcccagcagcagactcagctgaacctgcgctccctgctggtgaacccggagggccccaccctgatgcggctcaactccgtgcagagctcggagcggcccctgttcctggtgcacccaatcgagggctccaccaccgtgttccacagcctggcctcccggctcagcatccccacctatggcctgcagtgcacccgagctgcgccccttgacagcatccacagcctggctgcctactacatcgactgcatcaggcaggtgcagcccgagggcccctaccgcgtggccggctactcctacggggcctgcgtggcctttgaaatgtgctcccagctgcaggcccagcagagcccagcccccacccacaacagcctcttcctgttcgacggctcgcccacctacgtactggcctacacccagagctaccgggcaaagctgaccccaggctgtgaggctgaggctgagacggaggccatatgcttcttcgtgcagcagttcacggacatggagcacaacagggtgctggaggcgctgctgccgctgaagggcctagaggagcgtgtggcagccgccgtggacctgatcatcaagagccaccagggcctggaccgccaggagctgagctttgcggcccggtccttctactacaagctgcgtgccgctgagcagtacacacccaaggccaagtaccatggcaacgtgatgctactgcgcgccaagacgggtggcgcctacggcgaggacctgggcgcggactacaacctctcccaggtatgcgacgggaaagtatccgtccacgtcatcgagggtgaccaccgcacgctgctggagggcagcggcctggagtccatcatcagcatcatccacagctccctggctgagccacgcgtgagcgtgcgggagggctag

SEQ ID NO: 2 - RNA, 21 nt, Artificial Sequence
  Description of Artificial Sequence Synthetic oligonucleotide
cccugagaucccagcgcuguu

SEQ ID NO: 3 - RNA, 19 nt, Artificial Sequence
  Description of Artificial Sequence Synthetic oligonucleotide
uggagcguaucugugagaa

SEQ ID NO: 4 - RNA, 19 nt, Artificial Sequence
  Description of Artificial Sequence Synthetic oligonucleotide
ccauggagcguaucuguga

SEQ ID NO: 5 - RNA, 19 nt, Artificial Sequence
  Description of Artificial Sequence Synthetic oligonucleotide
ugacaucguccauucguuu

SEQ ID NO: 6 - RNA, 19 nt, Artificial Sequence
  Description of Artificial Sequence Synthetic oligonucleotide
gacgagagcaccuuugaug

SEQ ID NO: 7 - RNA, 19 nt, Artificial Sequence
  Description of Artificial Sequence Synthetic oligonucleotide
gagcguaucugugagaaac

SEQ ID NO: 8 - DNA, 23 bp, Artificial Sequence
  Description of Artificial Sequence Synthetic oligonucleotide
aannnnnnnnnnnnnnnnnnntt

SEQ ID NO: 9 - DNA, 51 bp, Homo sapiens
tttagaaatggctgtggctactcttmggcagtcagtggaggaagtgaaaaa

(19) United States
(12) Patent Application Publication
(10) Pub. No.: US 2014/0378388 A1
(43) Pub. Date: Dec. 25, 2014

(54) TREATMENT OF UTERINE LEIOMYOMATA

(71) Applicants: CYNTHIA C. MORTON, NEWTON, MA (US); STACEY L. EGGERT, WATERTOWN, MA (US)

(72) Inventors: CYNTHIA C. MORTON, NEWTON, MA (US); STACEY L. EGGERT, WATERTOWN, MA (US)

(73) Assignee: THE BRIGHAM AND WOMEN'S HOSPITAL, INC., BOSTON, MA (US)

(21) Appl. No.: 14/477,517

(22) Filed: Sep. 4, 2014

Related U.S. Application Data
(63) Continuation-in-part of application No. 14/237,622, filed on Sep. 15, 2014.
(60) Provisional application No. 61/521,204, filed on Aug. 8, 2011.

(51) Int. Cl. C12N 15/113; C12Q 1/68

(52) U.S. Cl. CPC C12N 15/113; C12Q 1/6827; C12N 2320/30
USPC 514/19.2; 514/44 A

(57) ABSTRACT

Embodiments herein provide a therapy for uterine leiomyomata (UL) in women using a fatty acid synthase (FAS) inhibitor. Additionally, an analysis method for evaluating the likelihood of women developing UL during their lifetime is provided.

### CROSS REFERENCE TO RELATED APPLICATION

This application is a continuation-in-part of co-pending U.S. application Ser. No. 14/237,622 filed on Feb. 7, 2014, which is a 35 U.S.C. §371 National Phase Entry of International Application No. PCT/US2012/049922 filed on Aug. 8, 2012, which designates the United States, and which claims the benefit under 35 U.S.C. §119(e) of U.S. Provisional Patent Application Ser. No. 61/521,204 filed on Aug. 8, 2011, the contents of each of which are incorporated herein by reference in its entirety.

### GOVERNMENT SUPPORT

This invention was made with Government support under Grant Nos.: HD046226 and HD060530 awarded by the National Institutes of Health. The Government has certain rights in the invention.

### SEQUENCE LISTING

The instant application contains a Sequence Listing which has been submitted electronically in ASCII format and is hereby incorporated by reference in its entirety. Said ASCII copy, created on Aug. 21, 2014, is named 043214-073023-US_SL.txt and is 12,090 bytes in size.

### FIELD

Disclosure herein relates to the treatment and management of uterine leiomyomata (UL), and the analysis of genetic predisposition to developing UL.

### BACKGROUND

Uterine leiomyomata (UL), commonly known as fibroids, are benign tumors of the uterine myometrium. They represent the most prevalent pelvic tumors in women, found in more than 70% of women of reproductive age. Approximately 20-25% of women with UL exhibit symptoms including menorrhagia, infertility, pelvic pain and a range of complications during pregnancy. The leading cause for hysterectomy in the United States, UL account for >30% of all hysterectomies and >40% of hysterectomies among women aged 45-64 years. Annual health care costs of UL are estimated at over two billion dollars, most of that cost associated with hysterectomies. Although UL pose a major public health problem, little is known about the molecular basis for these tumors and treatment options are limited.

Genes involved in UL have been discovered by cytogenetic analysis. Approximately 40% of UL have a non-random cytogenetic aberration and several subgroups are recognized, including t(12; 14)(q14-15; q23-24), del(7(q22q32), trisomy 12, rearrangements involving 6p21 and 10q22, and deletions of 1p and 3q. Cytogenetic abnormalities have been correlated with tumor size, location, and histology, which indicate that genetic events play a fundamental role in UL biology. Cytogenetic heterogeneity of UL underlies phenotypic differences and supports involvement of different pathways in tumor development.

Several factors predispose women to develop UL. Age, obesity, parity, and race have all been associated with prevalence of UL. Black women are disproportionately affected by UL, with incidence and prevalence rates at least three times greater than that in white women even after controlling for other known risk factors. Further, analyses of twin studies and familial aggregation indicate a genetic component to UL predisposition; first degree relatives of affected women have a 2.5-fold higher risk of developing UL and monozygotic twins' concordance for UL diagnosis is almost twice that of dizygotic twins'. Similarly, a study of a Finnish cohort found that monozygotic twins' concordance for being hospitalized for UL was twice that of dizygotic twins'. These findings support a genetic predisposition to develop UL but no genome-wide study of UL in white women has been reported. Several candidate gene association studies have been performed with limited success, although variants in the 5′ UTR of HMGA2, a gene involved in recurrent cytogenetic aberrations of UL and known to play a primary role, have been associated with UL diagnosis in a cohort of white sister pairs.

### SUMMARY

It is the objective of this disclosure to provide improved management, therapy and treatment for uterine leiomyomata (UL) in women.

It is also the objective of this disclosure to provide an analysis method for evaluating the likelihood of women developing UL during their lifetime. For example, UL can be recurrent.

Embodiments of this disclosure are based on the discovery of additional pathogenetic sequences that predispose women to develop UL. Specifically, women having UL tend to have the minor allele, A allele, at the rs4247357 SNP locus of chromosome 17 q25.3 region compared to women who do not have UL; these women tend to have the major allele, C allele. In addition, the women with UL and the minor allele have at least a two-fold increase in a fatty acid synthase protein (FAS) in their UL tissues compared to women with UL and the major allele. The FAS gene (FASN) is in linkage disequilibrium with the rs4247357 SNP locus.

Accordingly, the discovery provides a method of analyzing a woman's predisposition to UL by genotyping her genome at the rs4247357 SNP locus. Furthermore, the discovery provides a strategy of treatment and/or management of UL by inhibiting FAS protein activity and/or function and/or FASN expression.

In one embodiment, provided herein is a fatty acid synthase (FAS) inhibitor for use in the treatment and/or management of UL.

In another embodiment, provided herein is use of a FAS inhibitor in the manufacture of a medicament for the treatment and/or management of UL.

In one embodiment, provided herein is a composition for the treatment and/or management of UL, the composition comprising an inhibitor of FAS described herein and a pharmaceutical acceptable carrier. In one embodiment, the composition comprises at least one inhibitor of FAS described herein and a pharmaceutical acceptable carrier.

In one embodiment, provided herein is a composition comprising at least one inhibitor of FAS described herein and pharmaceutical acceptable carrier is formulated for administration by injection, infusion, instillation, vaginal suppository, cervical suppository, percutaneous implantation, or ingestion.

In one embodiment, provided herein is a method of treatment of UL in a female subject in need thereof comprising administering a therapeutically effective amount of FAS inhibitor described herein or a composition comprising an inhibitor of FAS described herein, whereby the UL is decreased in size in the subject relative to prior to the administration or whereby at least one symptom associated with UL is decreased in severity in the subject relative to prior to the administration. In one embodiment, the method further comprises determining the genotype at the rs4247357 SNP locus, wherein if the minor allele (A) is present, then proceeding with the treatment comprising FAS inhibitor.

In one embodiment, provided herein is a method of treatment of UL in a female subject in need thereof comprising determining the genotype at the rs4247357 SNP locus and administering a therapeutically effective amount of FAS inhibitor described herein or a composition comprising an inhibitor of a FAS described herein, whereby the UL is decreased in size in the subject relative to prior to the administration or whereby at least one symptom associated with UL is decreased in severity in the subject relative to prior to the administration.

In one embodiment, provided herein is a method of treatment of UL in a female subject in need thereof comprising providing a genetic sample from the subject, determining the subject's genotype at the rs4247357 SNP locus and administering a therapeutically effective amount of FAS inhibitor described herein or a composition comprising an inhibitor of a FAS described herein.

In one embodiment, treatment is performed when the minor allele (A) is present at the rs4247357 SNP locus. In another embodiment, treatment is performed even in the absence of the minor allele (A) at the rs4247357 SNP locus. In another embodiment, treatment is performed even in the absence of the minor allele (A) at the rs4247357 SNP locus and the female subject has developed UL.

In another embodiment, provided herein is a method of treatment of UL in a female subject in need thereof comprising administering a composition comprising an inhibitor of fatty acid synthase (FAS) and a pharmaceutical acceptable carrier.

In some embodiments, the FAS inhibitor can inhibit FAS activity or FASN gene expression, e.g., reducing the mRNA encoding FAS.

In some embodiments where the FAS inhibitor inhibits FAS activity, the FAS inhibitor is selected from the group consisting of a small molecule inhibitor, a polyphenol and a dietary compound.

In some embodiments where the FAS inhibitor inhibits FAS activity, the inhibitor of FAS activity is selected from the group consisting of(S)—((S)-1-((2S,3S)-3-hexyl-4-oxooxetan-2-yl)tridecan-2-yl) 2-formamido-4-methylpentanoate, 3-Carboxy-4-octyl-2-methylenebutyrolactone, trans-4-Carboxy-5-octyl-3-methylenebutyrolactone (C75), cerulenin, C93 (FAS93), FAS31, C247, GSK837149A, platensimycin, 3-aryl-4-hydroxyquinolin-2(1H)-one scaffold (MERCK), bisamide scaffold (AstraZeneca), epigallocatechin, luteolin, taxifolin, kaempferol, quercetin, apigenin, catechin, soy protein and monounsaturated fatty acid oleic acid (18:1, n-9).

In other embodiments where the FAS inhibitor inhibits FAS activity, the inhibitor of FAS activity is selected from the group consisting of polyhydroxylated compounds of US 2010/0190856, cerulenin compounds of U.S. Pat. No. 5,981,575, spirocyclic piperidines of WO2012/064642, and compounds of US 20100022630.

In some embodiments where the FAS inhibitor inhibits FASN expression, the inhibitor of FASN expression is selected from a small molecule and a nucleic acid.

In some embodiments where the FAS inhibitor inhibits FASN expression, the FAS inhibitor is a FAS specific RNA interference agent, or a vector encoding a FAS specific RNA interference agent.

In one embodiment, the RNA interference agent is a double stranded RNA (dsRNA).

In some embodiments, the RNA interference agent comprises one or more of the nucleotide sequences of CCCUGAGAUCCCAGCGCUGUU (SEQ.ID.NO:2), UGGAGCGUAUCUGUGAGAA (SEQ.ID.NO:3), CCAUGGAGCGUAUCUGUGA (SEQ.ID.NO:4), UGACAUCGUCCAUUCGUUU (SEQ.ID.NO:5), GACGAGAGCACCUUUGAUG (SEQ.ID.NO:6), and GAGCGUAUCUGUGAGAAAC (SEQ.ID.NO:7).

In one embodiment, the FAS inhibitor is formulated for administration by injection, infusion, instillation, vaginal suppository, cervical suppository, percutaneous implantation, or ingestion. In another embodiment, the composition comprising the FAS inhibitor is formulated for administration by injection, infusion, instillation, vaginal suppository, cervical suppository, percutaneous implantation, or ingestion.

In one embodiment, the subject is female. In another embodiment, the subject has been diagnosed with uterine leiomyomata.

In one embodiment, the female subject has the minor SNP allele, adenine (A) at rs4247357 in chromosome 17. In another embodiment, the subject is homozygous for the minor (A) SNP allele at rs4247357 in chromosome 17. In another embodiment, the subject is heterozygous for the minor (A) SNP allele at rs4247357 in chromosome 17.

In one embodiment, provided herein is a method comprising providing a sample from a female subject for assessing the increased likelihood of developing uterine leiomyomata (UL); determining the single nucleotide polymorphism (SNP) allelic genotype at rs4247357 at chromosome 17, wherein a homozygosity (AA) and heterozygosity (AC) for the minor SNP allele indicates an increased likelihood of developing UL.

In one embodiment, the sample from the female subject is a tissue sample comprising deoxyribonucleic acid (DNA). In one embodiment, any source of constitutional or likely even tumor DNA would be acceptable for genotyping. In another embodiment, a tissue sample adjacent to a fibroid tumor is used for genotyping. In some embodiments, the genetic sample from a female subject is selected from the group consisting of a blood sample, a saliva sample, a skin sample, a hair bulb and an epithelial sample.

In one embodiment, the female subject has reached puberty. In another embodiment, the female subject has not reached menarche.

In one embodiment, the female subject is between the ages of 7-70.

In one embodiment, the female subject has not entered perimenopause or menopause. In another embodiment, the female subject has entered perimenopause or menopause.

In one embodiment, the female subject is on hormone replacement therapy.

In one embodiment, the female subject has at least one first and/or second degree relative that has or has had UL.

In one embodiment, the (SNP) allelic genotype is performed by hybridization-based methods or by enzyme-based methods. In one embodiment, the hybridization-based methods comprise SNP microarrays. In another embodiment, the SNP allelic genotype is performed by DNA sequencing.

### BRIEF DESCRIPTION OF THE DRAWINGS

FIG. 1 shows the summary of the “Finding Genes for Fibroids” (FGFF) sib-pair linkage analysis by chromosome. Two peaks have significant LOD scores (>3.6) and five peaks have suggestive LOD scores (>2.0). Linkage peak boundaries and the highest LOD score found under each peak are defined in the inserted table.

FIG. 2 shows the quantile-quantile plot of meta-analysis results. P-values observed in the study are compared to p-values expected under the null hypothesis. Top SNPs in the 17q25.3 region are circled.

FIG. 3 shows the candidate region on chromosome 17 containing significantly associated markers. P-values for the Women's Genome Health Study (WGHS) genotyped SNPs are indicated by diamonds, p-values for WGHS imputed SNPs are indicated by circles and p-values for SNPs included in the meta-analysis are indicated by triangles.

FIG. 4 shows the FAS protein levels in myometrium and uterine leiomyomata (UL) from matched samples. Representative tissue cores are shown from myometrium (left) and UL (right) tissues. Significant increase compared to myometrium was determined by t test: *p=0.004.

FIG. 5A shows the FAS protein levels in myometrium and UL from matched samples stratified by rs4247357 genotype: AA, AC or CC.

FIG. 5B shows the FAS protein levels in myometrium and UL from all samples stratified by rs4247357 genotype: AA, AC or CC.

FIG. 6 shows the position of the FGFF linkage peak at 12q14 comprising HMGA2.

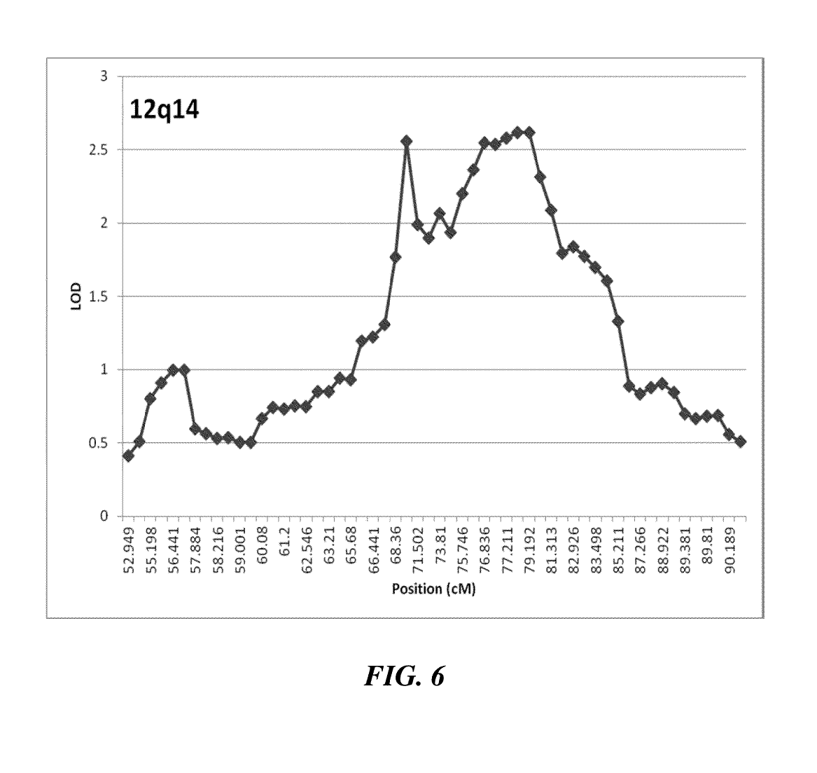

FIG. 7A shows the quantile-quantile plot of the WGHS Genome-Wide Association Studies (GWAS) results. P-values observed in the studies are compared to p-values expected under the null hypothesis.

FIG. 7B shows the quantile-quantile plot of the Australian Genome-Wide Association Studies (GWAS) results. P-values observed in the studies are compared to p-values expected under the null hypothesis.

FIG. 8 shows the HapMap generated linkage disequilibrium (LD) plot of the candidate region on chromosome 17.

FIG. 9 shows the position of the FGFF linkage peak at 17q25 comprising FASN.

FIG. 10 shows the ratio of FAS protein expression in matched UL/myometrium samples grouped by rs4247357 genotype. Each bar represents the ratio from one woman.

FIG. 11A shows the mRNA expression by qPCR of FASN, CCDC57, SLC16A3 and three genes in direct proximity (DUS1L, (CSNK1D, NARF) in myometrium and matched UL samples.

FIG. 11B shows the mRNA expression by qPCR of FASN, (CCD57, SLC16A3 and three genes in direct proximity (DUS1L, CSNK1D, NARF) in myometrium with the major (CC) and minor (AA) allele of rs4247357.

FIG. 11C shows the mRNA expression by qPCR of FASN, CCDC57, SLC16A3 and three genes in direct proximity (DUS1L, CSNK1D, NARF) in UL with the major (CC) and minor (AA) allele of rs4247357.

### DETAILED DESCRIPTION

Unless otherwise explained, all technical and scientific terms used herein have the same meaning as commonly understood by one of ordinary skill in the art to which this disclosure belongs. Definitions of common terms in genetics and molecular biology may be found in Benjamin Lewin, Genes IX, published by Jones & Bartlett Publishing, 2007 (ISBN-13: 9780763740634); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8). Further, unless otherwise required by context, singular terms shall include pluralities and plural terms shall include the singular.

Unless otherwise stated, embodiments of the present disclosure described herein were performed using standard procedures known to one skilled in the art, for example, in Current Protocols in Human Genetics (CPHG) (Haines, et al., ed., John Wiley and Sons, Inc.), Maniatis et al., Molecular Cloning: A Laboratory Manual, Cold Spring Harbor Laboratory Press, Cold Spring Harbor, N.Y., USA (1982); Sambrook et al., Molecular Cloning: A Laboratory Manual (2^{nd} ed.), Cold Spring Harbor Laboratory Press, Cold Spring Harbor, N.Y., USA (1989); Davis et al., Basic Methods in Molecular Biology, Elsevier Science Publishing, Inc., New York, USA (1986); Current Protocols in Molecular Biology (CPMB) (Ausubel, et al. ed., John Wiley and Sons, Inc.), Current Protocols in Immunology (CP) (Coligan, et al., ed. John Wiley and Sons, Inc.), Current Protocols in Cell Biology (CPCB) (Bonifacino et al., ed., John Wiley and Sons, Inc.), Culture of Animal Cells: A Manual of Basic Technique by Freshney, Wiley-Liss; 5th ed. (2005), Methods in Molecular Biology, Vol. 180, and Methods in Molecular Biology, Vol. 203, 2003, Transgenic Mouse, edited by Hofker and Deursen, ed. which are all incorporated herein by reference in their entireties.

It should be understood that embodiments of the present disclosure described herein are not limited to the particular methodology, protocols, and reagents, etc., described herein and as such may vary. The terminology used herein is for the purpose of describing particular embodiments only, and is not intended to limit the scope of the present inventions, which is defined solely by the claims.

Other than in the operating examples, or where otherwise indicated, all numbers expressing quantities of ingredients or reaction conditions used herein should be understood as modified in all instances by the term “about.”

All patents and publications identified are expressly incorporated herein by reference for the purpose of describing and disclosing, for example, the methodologies described in such publications that might be used in connection with the present inventions. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

Embodiments described herein are based on the discovery that there is a higher level of the human fatty acid synthase protein (FAS) in the uterine leiomyomata (UL) of women having the minor allele A at the single nucleotide polymorphism (SNP) rs4247357 on chromosome 17q25.3 region compared to the UL of women having the major allele C, and that the FAS gene (FASN) is in linkage disequilibritun with the rs4247357 SNP locus. Accordingly, the discovery provides a method of treatment and/or management of UL by inhibiting FAS protein activity and/or function, and/or inhibiting the expression of FASN. In addition, the discovery provides a method of evaluating a woman's predisposition or likelihood of developing UL by genotyping her genome at the rs4247357 SNP locus.

UL are benign smooth muscle neoplasms or tumors in the uterus. They are by far the most prevalent pelvic tumors in women of reproductive age. They are mostly benign, but may lead to excessive menstrual bleeding (menorrhagia), bleeding between periods, often cause anemia, may lead to infertility, pelvic pressure, stress urinary incontinence and ureteral obstruction. These complications have associated morbidities and are the most common indication for hysterectomies. They are found in nearly half of women over age 40. Because of these complications and prevalence, the condition poses a major public health problem.

The inventors have found a genetic component to UL predisposition that is supported by analyses of ethnic predisposition, twin studies, and familial aggregation. A genome-wide SNP linkage panel was genotyped and analyzed in 261 Caucasian (white) UL sister pair families from the “Finding Genes for Fibroids study” (FGFF). Two significant linkage regions were detected in 10p11 (LOD=4.15) and 3p21 (LOD=3.73) while five additional linkage regions were identified with LOD scores >2.00 in 2q37, 5p13, 11p15, 12q14 and 17q25. Genome-Wide Association Studies (GWAS) were performed in two independent cohorts of white women and a meta-analysis was conducted. One SNP was identified with a p-value that reached genome-wide significance (rs4247357, P=3.05E-08, odds ratio (OR)=1.299). The candidate SNP is under a linkage peak and in a block of linkage disequilibrium in 17q25.3 which spans the genes fatty acid synthase (FASN), coiled-coil domain containing 57 (CCDC57) and solute carrier family 16, member 3 (SLC16A3). By tissue microarray immunohistochemistry, the inventors found FAS protein levels elevated (3-fold) in UL when compared to matched myometrial tissue. FAS transcripts and/or protein levels are up-regulated in various neoplasms and implicated in tumor cell survival. This is the first observation of elevated FAS protein association with the benign tumors of UL. FASN represents the initial UL risk allele identified in white women by a genome-wide, unbiased approach.

Accordingly, in one embodiment, provided herein is a fatty acid synthase (FAS) inhibitor for use in the treatment and/or management of UL.

In another embodiment, provided herein is a use of a FAS inhibitor in the manufacture of a medicament for the treatment and/or management of UL.

In one embodiment, provided herein is a composition for the treatment and/or management of UL, the composition comprising an inhibitor of FAS described herein and a pharmaceutical acceptable carrier. In another embodiment, the composition comprises at least one inhibitor of FAS described herein and a pharmaceutical acceptable carrier. In another embodiment, the composition comprises more than one inhibitor of FAS described herein and a pharmaceutical acceptable carrier. When more than one FAS inhibitor is present in the composition, the FAS inhibitors are different in their activities. For example, the composition comprises an inhibitor of FAS activity/function and another inhibitor of FASN expression.

In one embodiment, provided herein is a method of treatment of UL in a subject in need thereof comprising administering a therapeutically effective amount of FAS inhibitor described herein or a composition comprising an inhibitor of FAS described herein. In one embodiment, the UL is decreased in size in the subject relative to prior to the administration. In another embodiment, at least one clinical symptom associated with UL is decreased relative to prior to the administration. In some embodiment, a clinical symptom associated with UL is selected from the group consisting of menorrhagia, bleeding between periods, anemia, infertility, bowel obstruction or constipation, pelvic pressure, stress urinary incontinence and ureteral obstruction for selecting a female subject for therapy.

In another embodiment, provided herein is a method of treatment of UL in a female subject in need thereof comprising administering a composition comprising an inhibitor of FAS and a pharmaceutical acceptable carrier.

One embodiment of the treatment method further comprises determining the genotype at the rs4247357 SNP locus, wherein if the minor allele (A) is present, then the treatment comprising FAS inhibitor is administered.

In one embodiment, provided herein is a method of treatment of UL in a female subject in need thereof comprising determining the female subject's genotype at the rs4247357 SNP locus and administering a therapeutically effective amount of FAS inhibitor described herein or a composition comprising an inhibitor of FAS described herein, whereby the UL is decreased in size in the subject relative to prior to the administration or whereby at least one symptom associated with UL is decreased in severity in the subject relative to prior to the administration.

As used herein, the term “decreased in severity” when used in relation to symptoms associated with UL refers to a decrease in the amount of any of the symptoms (e.g., of menorrhagia, bleeding between periods, anemia, infertility, pelvic pressure, stress urinary incontinence, bowel obstruction, constipation and ureteral obstruction) by at least 5%, at least 10%, at least 20%, at least 30%, at least 40%, at least 50%, at least 60%, at least 70%, at least 80%, at least 90%, or at least 1-fold lower, at least 2-fold lower, at least 5-fold lower, at least 10 fold lower, at least 100 fold lower, at least 1000-fold lower, or more compared to the symptoms prior to treatment with the FAS inhibitor.

In one embodiment, provided herein is a method of treatment of UL in a female subject in need thereof comprising providing a genetic sample from the subject, determining the subject's genotype at the rs4247357 SNP locus and administering a therapeutically effective amount of FAS inhibitor described herein or a composition comprising an inhibitor of FAS described herein, whereby the UL is decreased in size in the subject relative to prior to the administration or whereby at least one symptom associated with UL is decreased in severity in the subject relative to prior to the administration.

In one embodiment, treatment is performed when the minor allele (A) is present at the rs4247357 SNP locus in the female subject. In another embodiment, treatment is performed even in the absence of the minor allele (A) at the rs4247357 SNP locus. In another embodiment, treatment is performed even in the absence of the minor allele (A) at the rs4247357 SNP locus and the subject presents with symptoms associated with UL and/or has UL.

In one embodiment, as used herein, the term “treat’ or treatment” refers to reducing or alleviating at least one adverse clinical symptom associated with UL, e.g., menorrhagia. In another embodiment, the term “treat’ or treatment” also refers to slowing or reversing the progression or growth of the UL.

As used herein, the term “a therapeutically effective amount” refers to an amount sufficient to achieve the intended purpose of treating UL. The amount is that which is safe and sufficient to treat, lesson the likelihood of, or delay the development of a UL. The amount can thus cure or result in amelioration of the symptoms of the UL, slow the course of UL disease progression, slow or inhibit a symptom of a UL, slow or inhibit the establishment of secondary symptoms of a UL or inhibit the development of a secondary symptom of a UL. For example, an effective amount of a FAS inhibitor that inhibits UL further growth, causes a reduction in size or even completely halts UL growth, shrinks the sizes of UL, even complete regression of UL, reduces clinical symptoms associated with UL such as menorrhagia, infertility, anemia, incontinence and ureteral, intestine or bowel obstruction. An effective amount for treating or ameliorating UL is an amount of FAS inhibitor sufficient to result in a reduction or complete removal of the symptoms of the disorder, disease, or medical condition. The effective amount of a given FAS inhibitor will vary with factors such as the nature of the agent, the route of administration, the size and species of the subject to receive the FAS inhibitor, and the purpose of the administration. Thus, it is not possible or prudent to specify an exact “effective amount”. However, for any given case, an appropriate “effective amount” can be determined by a skilled artisan according to established methods in the art using only routine experimentation.

As used herein, the terms “FAS inhibitor” or “inhibitor of FAS” are used interchangeably and refer to the inhibition FAS activity, i.e., the enzymatic function of the protein FAS, or inhibition of FASN expression, i.e., transcription of F ASN.

By “inhibition of FASN expression” is meant that the amount of expression of FASN is at least 5% lower in a population of cells treated with a FAS inhibitor, than a comparable, control population of cells, wherein no FAS inhibitor is present. It is preferred that the percentage of FASN expression in a FAS inhibitor treated population is at least 5% lower, at least 10% lower, at least 20% lower, at least 30% lower, at least 40% lower, at least 50% lower, at least 60% lower, at least 70% lower, at least 80% lower, at least 90% lower, at least 1-fold lower, at least 2-fold lower, at least 5-fold lower, at least 10-fold lower, at least 100-fold lower, at least 1000-fold lower, or more than a comparable control treated population in which no FAS inhibitor is added.

By “inhibits FAS activity” is meant that the amount of functional enzymatic activity of FAS is at least 5% lower in a population of cells treated with a FAS inhibitor, than a comparable, control population of cells, wherein no FAS inhibitor is present. It is preferred that the percentage of FAS activity in a FAS-inhibitor treated population is at least 5% lower, at least 10%/o lower, at least 20% lower, at least 30% lower, at least 40% lower, at least 50% lower, at least 60% lower, at least 70% lower, at least 80% lower, at least 90% lower, at least 1-fold lower, at least 2-fold lower, at least 5-fold lower, at least 10-fold lower, at least 100-fold lower, at least 1000-fold lower, or more than a comparable control treated population in which no FAS inhibitor is added. At a minimum, FAS activity can be assayed by determining the amount of FAS expression at the protein or mRNA levels, using techniques standard in the art. Alternatively, or in addition, FAS activity can be determined using any FAS enzyme assay known in the art. For example, FAS assays as described in J. A. Moibi, et al., 2000, J. Anim. Sci., 78:2383-2392; N. W. Bays, et al., 2009, J Biomol. Screen 14:6 636-642; V. Sabbisetti et al., 2009, PLoS ONE 4(6): c5877; and Ji-Nong Li et al., 2001, Cancer Res., 61; 1493. Alternatively, the amount of free fatty acid can be quantitated as the indication of FAS activity (end product analysis of an enzymatic reaction). Commercial kits for measuring free fatty acids are available, e.g., Free Fatty Acid Quantification Kit (catalog#ab65341) by ABCAM® or the PATHSCAN® Total Fatty Acid Synthase Sandwich ELISA Kit catalog #7689 by CELL SIGNALING TECHNOLOGY®. A decrease in FAS activity or free fatty acid synthesis of at least 10% is indicative of a compound being a FAS inhibitor.

As used herein, the term “gene” means the nucleic acid sequence which is transcribed (DNA) and translated (mRNA) into a polypeptide in vitro or in vivo when operably linked to appropriate regulatory sequences. The gene may or may not include regions preceding and following the coding region, e.g. 5′ untranslated (5′ UTR) or “leader” sequences and 3′ UTR or “trailer” sequences, as well as intervening sequences (introns) between individual coding segments (exons).

A “nucleic acid”, as described herein, can be RNA or DNA, and can be single or double stranded, and can be selected, for example, from a group including: nucleic acid encoding a protein of interest, oligonucleotides, nucleic acid analogues, for example peptide-nucleic acid (PNA), pseudo-complementary PNA (pc-PNA), locked nucleic acid (LNA) etc. Such nucleic acid sequences include, for example, but are not limited to, nucleic acid sequence encoding proteins, for example that act as transcriptional repressors, antisense molecules, ribozymes, small inhibitory nucleic acid sequences, for example but are not limited to RNAi, shRNAi, siRNA, micro RNAi (mRNAi), antisense oligonucleotides, etc.

In one embodiment, the FAS inhibitor can either inhibit FAS activity, i.e., the enzymatic function of the FAS protein, or inhibit FASN gene expression.

In humans, FAS is an enzyme that is encoded by FASN. It is a multi-enzyme protein that catalyzes fatty acid synthesis. It is not a single enzyme but a whole enzymatic system composed of two identical 272 kDa multifunctional polypeptides, in which substrates are handed from one functional domain to the next. Mammalian FAS consists of a homodimer of two identical protein subunits, in which three catalytic domains in the N-terminal section (-ketoacyl synthase (KS), malonyl/acetyltransferase (MAT), and dehydrase (DH)), are separated by a core region of 600 residues from four C-terminal domains (enoyl reductase (ER), -ketoacyl reductase (KR), acyl carrier protein (ACP) and thioesterase (TE)). FAS's main function is to catalyze the synthesis of palmitate from acetyl-CoA and malonyl-CoA, in the presence of NADPH, into long-chain saturated fatty acids.

FAS has been investigated as a possible oncogene. FAS is up-regulated in breast cancers, and as well as being an indicator of poor prognosis may also be worthwhile as a chemotherapeutic target (30-40). FAS may also be involved in the production of an endogenous ligand for the nuclear receptor PPARalpha, the target of the fibrate drugs for hyperlipidemia, (41) and is being investigated as a possible drug target for treating metabolic syndrome (42).

In some cancer cell lines, this protein has been found to be fused with estrogen receptor alpha (ER-alpha), in which the N-terminus of FAS is fused in-frame with the C-terminus of ER-alpha.

FAS inhibitors that inhibit FAS activity are known in the art, see U.S. Pat. No. 5,981,575, US 2010/0190856 and 2010/0022630, and WO2012/064642, and R. Flavin et al., (2011) (43) as non-limiting examples and these references are incorporated herein by reference in their entirety. FAS inhibitors have been used in breast cancer animal models and several xenograft models, and the results indicate that FAS inhibitors can delay development and growth of the tumors (38,39). FAS inhibitors can be divided into several categories: small molecule inhibitor, naturally occurring polyphenols and dietary compound, see R. Flavin et al., (2011) (43). Non-limiting examples of small molecule FAS inhibitors include cerulenin, C75 (catalog #341325, CALBIOCHEM®), ORLISTAT (aka tetrahydrolipstatin), C93 (FAS93), FAS31, C247, GSK837149A, platensimycin, 3-aryl-4-hydroxyquinolin-2(1H)-one scaffold (MERCK), bisamide scaffold (ASTRAZENECA) and functional analogs thereof. Non-limiting examples of naturally occurring polyphenols include epigallocatechin, luteolin, taxifolin, kaempferol, quercetin and apigenin. Non-limiting examples of dietary compound that can inhibit FAS include catechin, soy protein and monounsaturated fatty acid oleic acid (18:1, n-9).

In one embodiment, the FAS inhibitor that inhibits FAS activity includes but is not limited to the FAS inhibitor selected from the group consisting of a small molecule inhibitor, a polyphenol and a dietary compound.

As used herein, the term “small molecule” refers to a chemical agent including, but not limited to, peptides, peptidomimetics, amino acids, amino acid analogs, polynucleotides, polynucleotide analogs, aptamers, nucleotides, nucleotide analogs, organic or inorganic compounds (i.e., including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds.

As used herein, the term “dietary compound” refers to a compound that is related to food available to or eaten by a female subject. For example, the “dietary compound” is derived, reduced, modified or extracted from food available to or eaten by a subject.

In one embodiment, the small molecule FAS inhibitor that inhibits FAS activity includes but is not limited to the small molecule selected from the group consisting of (S)—((S)-1-((2S,3S)-3-hexyl-4-oxooxetan-2-yl)tridecan-2-yl) 2-formamido-4-methylpentanoate (orlistat), 3-Carboxy-4-octyl-2-methylenebutyrolactone, trans-4-Carboxy-5-octyl-3-methylenebutyrolactone (C75), cerulenin, C93 (FAS93), FAS31, C247, GSK837149A, platensimycin, 3-aryl-4-hydroxyquinolin-2(1H)-one scaffold (MERCK), and bisamide scaffold (AstraZeneca).

In one embodiment, the polyphenol that inhibits FAS activity includes but is not limited to the polyphenol selected from the group consisting of epigallocatechin, luteolin, taxifolin, kaempferol, quercetin and apigenin. Functional analogs thereof of these polyphenols are also contemplated.

In one embodiment, the dietary compound that inhibits FAS activity includes but is not limited to the dietary compound selected from the group consisting of catechin, soy protein and monounsaturated fatty acid oleic acid (18:1, n-9).

In one embodiment, the FAS inhibitor that inhibits FAS activity includes but is not limited to the inhibitor of FAS activity selected from the group consisting of polyhydroxylated compounds of US 2010/0190856, cerulenin compounds of U.S. Pat. No. 5,981,575, spirocyclic piperidines of WO2012/064642, and compounds of US 2010/0022630.

Accordingly, in one embodiment, the composition described herein having more than one FAS inhibitor comprises a mixture of FAS inhibitors, e.g., a small molecule inhibitor, a polyphenol and a dietary compound, all of which inhibit FAS activity. For example, the composition having more than one FAS inhibitor comprises at least one small molecule FAS inhibitor and at least one polyphenol. In one embodiment, the composition having more than one FAS inhibitor comprises at least one small molecule FAS inhibitor, at least one dietary compound that inhibits FAS activity and at least one polyphenol that inhibits FAS activity. Non-limiting examples include C75 and catechin, orlistat, taxifolin and catechin, and cerulenin and monounsaturated fatty acid oleic acid.

In one embodiment, the FAS inhibitor inhibits FASN expression. In one embodiment, the inhibitor of FASN expression is selected from a small molecule and a nucleic acid. In another embodiment, the inhibitor of FASN expression is a FAS specific RNA interference agent, or a vector encoding a FAS specific RNA interference agent.

As used herein, the term “vector” refers to a nucleic acid molecule capable of transporting another nucleic acid to which it has been linked. One type of vector is a “plasmid”, which refers to a circular double stranded DNA loop into which additional nucleic acid segments can be ligated. Another type of vector is a viral vector, wherein additional nucleic acid segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors”, or more simply “expression vectors.” In general, expression vectors of utility in recombinant DNA techniques are often in the form of plasmids. In the present specification, “plasmid” and “vector” can be used interchangeably as the plasmid is the most commonly used form of vector. However, the disclosure herein is intended to include such other forms of expression vectors, such as viral vectors (e.g., replication defective retroviruses, lentiviruses, adenoviruses and adeno-associated viruses), which serve equivalent functions. In one embodiment, lentiviruses are used to deliver one or more siRNA molecule of the treatment described herein.

In one embodiment, the RNA interference agent is a double stranded RNA (dsRNA) or a short hairpin dsRNA. In one embodiment, the dsRNA is synthesized in vitro, for example, by the annealing of two complementary strands of RNAs that are independently synthesized in vitro. Alternatively, the dsRNA is synthesized in vivo, i.e., the dsRNA is expressed in the cells with a vector encoding a FAS specific dsRNA.

In one embodiment, the RNA interference agent comprises one or more of the nucleotide sequences of CCCUGAGAUCCCAGCGCUGUU (SEQ.ID.NO:2), UGGAGCGUAUCUGUGAGAA (SEQ.ID.NO:3), CCAUGGAGCGUAUCUGUGA (SEQ.ID.NO:4), UGACAUCGUCCAUUCGUUU (SEQ.ID.NO:5), GACGAGAGCACCUUUGAUG (SEQ.ID.NO:6), and GAGCGUAUCUGUGAGAAAC (SEQ.ID.NO:7).

In one embodiment, the FAS inhibitor formulated for administration by injection, infusion, instillation, vaginal suppository, cervical suppository, percutaneous implantation, or ingestion. In another embodiment, the composition comprising the FAS inhibitor is formulated for administration by injection, infusion, instillation, vaginal suppository, cervical suppository, percutaneous implantation, or ingestion.

In one embodiment, the subject is a human female subject. In another embodiment, the subject has been diagnosed with UL. In another embodiment, the subject presents with symptoms of UL. In some embodiments, the subject presents at least one symptom selected from the group consisting of menorrhagia, bleeding between periods, anemia, infertility, pelvic pressure, stress urinary incontinence and ureteral obstruction.

UL are characterized by benign, smooth muscle tumors of the uterus. These tumors can range in size. When tumors are present, especially larger than one centimeter in diameter, the uterus is irregularly enlarged and usually somewhat asymmetrical. It may be tender and may assume very large sizes. Unlike the soft uterus containing a pregnancy or adenomyosis, the fibroid uterus is very firm. Clinical symptoms of UL include but are not limited to an enlarged, irregularly shaped, firm uterus that may or may not be tender, menorrhagia, bleeding between periods, anemia, infertility, pelvic pressure, stress urinary incontinence and ureteral obstruction. Confirmation of diagnosis can be performed with any of the known methods which include but are not limited to ultrasound, MRI and CT scanning, laparoscopy and histology. A skilled gynecologist would be able to make a differential diagnosis of the condition.

In one embodiment, the female subject being treated has the minor SNP allele, adenine (A) at rs4247357 in chromosome 17. In another embodiment, the subject is homozygous for the minor (A) SNP allele at rs4247357 in chromosome 17. In another embodiment, the subject is heterozygous for the minor (A) SNP allele at rs4247357 in chromosome 17.

The SNP allele analysis can be performed by any method known in the art. For example, by hybridization-based methods which include dynamic allele-specific hybridization, allele specific oligonucleotide (ASO) hybridization, molecular beacons and SNP microarrays; by enzyme-based methods which include restriction fragment length polymorphisms (RFLPs), PCR-based methods, Flap endonuclease, primer extension, 5′-nuclease, PCR with Sanger sequencing and oligonucleotide ligase assay; and by other post-amplification methods based on physical properties of DNA such as single strand conformation polymorphism, temperature gradient gel electrophoresis, denaturing high performance liquid chromatography, high-resolution melting of the entire amplicon, use of DNA mismatch-binding proteins and SNPlex. See Xiaofeng Zhou and David T. W. Wong, 2007, Methods in Molecular Biology, Vol. 396, pages 295-314. This reference is incorporated herein by reference in its entirety.

In one embodiment, any sample from the female subject that contains DNA can be used for genotyping. In practice, a tissue sample is obtained with minimal or no invasive procedure, meaning the tissue is obtained from a bodily area/part that is easily accessible and the tissue is easily dislodged from the body. In some embodiments, the tissue sample from the subject is selected from the group consisting of a blood sample, a saliva sample, a skin sample, a hair bulb and an epithelial sample.

In one embodiment, the female subject being treated has reached puberty. In another embodiment, the female subject being treated has not reached menarche.

As used herein, the term “puberty” refers to the stage of adolescence in which a subject becomes physiologically capable of sexual reproduction. For a female subject, the subject would have undergone secondary sexual development that is manifested in pubic hair at/near the genital area, axillary (arm pit) hair, and development of breasts.

As used herein, the term “menarche” refers to the first menstrual cycle, or first menstrual bleeding, in human females.

In one embodiment, the female subject being treated is between the ages of 7-70. In other embodiments, the subject being treated is between the ages of 7-65, 7-60, 7-55, 7-50, 7-45, 7-40, 7-35, 10-70, 10-65, 10-60, 10-55, 10-50, 10-45, 10-40, 10-35, 12-70, 12-65, 12-60, 12-55, 12-50, 12-45, 12-35, or 12-30, and includes all intermediate ranges between 7-70.

In one embodiment of the therapeutic method, the female subject being treated has not entered perimenopause or menopause. In another embodiment, the subject being treated has entered perimenopause or menopause.

In one embodiment of the therapeutic method, wherein the female subject being treated is or was on hormone replacement therapy.

In one embodiment of the therapeutic method, the female subject being treated has at least one first and/or second degree relative that has or has had UL.

The inventors have shown that human females having the minor SNP allele at at rs4247357 on chromosome 17 are predisposed to develop UL and have higher levels of FAS in the UL. This indicates that there is a genetic component or predisposition to developing UL.

Accordingly, this discovery provides a method of predicting the likelihood of a human female having UL in her life time. The value of this prediction allows better preemptive gynecological care, especially during her reproductive years.

In one embodiment, provided herein is a method comprising providing a genetic sample from a human female subject for assessing the increased likelihood of developing UL; determining the SNP allelic genotype at rs4247357 on chromosome 17, wherein a homozygosity (AA) and heterozygosity (AC) for the minor SNP allele indicates an increased likelihood of developing UL.

In one embodiment of the method of assessing the likelihood of UL, the genetic sample from the female subject is a tissue sample comprising DNA. Basically any genetic or tissue sample from the female subject that contains DNA can be used for genotyping. In practice, a genetic or tissue sample is obtained with minimal or no invasive procedure, meaning the tissue is obtained from a bodily area/part that is easily accessible and the tissue is easily dislodged from the body. (E.g., a scraping of the epithelial cells from the inner lining of the mouth). This is commonly achieved with a cotton swab. In some embodiments, the genetic sample from a female subject is selected from the group consisting of a blood sample, a saliva sample, a skin sample, a hair bulb and an epithelial sample. The hair bulb is the root of the hair ends that is expanded that fits like a cap over the hair papilla at the bottom of the hair follicle. The hair bulb is whiter in color and softer in texture than the shaft, and is lodged in a follicular involution of the epidermis called the hair follicle.

It is known that UL tend to grow under the influence of estrogen, and regress when the estrogen levels are reduced. Thus, growth frequently occurs during pregnancy, followed by regression following delivery. After the onset of menopause, fibroids generally regress. High-dose birth control pills, by virtue of their high estrogen content, can cause UL to grow larger. Low-dose birth control pills, in contrast, leave circulating estrogen levels the same (or reduced) and do not stimulate fibroid growth.

Accordingly, in one embodiment of the method of assessing the likelihood of UL, the female subject has reached puberty. In another embodiment, the female subject has not reached menarche.

In one embodiment of the method of assessing the likelihood of UL, the female subject is between the ages of 7-70. In other embodiments, the female subject being treated is between the ages of 7-65, 7-60, 7-55, 7-50, 7-45, 7-40, 7-35, 10-70, 10-65, 10-60, 10-55, 10-50, 10-45, 10-40, 10-35, 12-70, 12-65, 12-60, 12-55, 12-50, 12-45, 12-35, or 12-30, and includes all intermediate ranges between 7-70.

In one embodiment of the method of assessing the likelihood of UL, the female subject has not entered perimenopause or menopause. In another embodiment, the subject has entered perimenopause or menopause.

As used herein, the term “menopause” refers to the permanent end of menstruation and fertility, and is defined as occurring 12 months after the last menstrual period.

As used herein, the term “perimenopause” refers to the interval in which a woman's body makes a natural shift from more-or-less regular cycles of ovulation and menstruation toward permanent infertility, or menopause. “Perimenopause” is also called the menopausal transition. The transition is characterized by menstrual periods becoming irregular—longer, shorter, heavier or lighter, sometimes more and sometimes less than 28 days apart. The female may also experience menopause-like symptoms, such as hot flashes, sleep problems and vaginal dryness.

In one embodiment of the method of assessing the likelihood of UL, the female subject is on hormone replacement therapy.

As used herein, the term “hormone replacement therapy” refers to the use of synthetic or natural female hormones to make up for the decline or lack of natural hormones produced in a woman's body. HRT is sometimes referred to as estrogen replacement therapy (ERT), because the first medications that were used in the 1960s for female hormone replacement were estrogen compounds. Generally, hormone replacement therapy has been prescribed for two primary purposes: preventive treatment against osteoporosis and heart disease, and relief of physical symptoms associated with menopause.

In one embodiment of the method of assessing the likelihood of UL, the female subject has at least one first and/or second degree relative that has or has had UL.

As used herein, the term “first degree relative” refers to immediate family members of the referenced female subject, e.g., a mother, sister or daughter.

As used herein, the term “second degree relative” refers to family members once removed from the referenced female subject, i.e., separated by one generation. For example, grandmother, aunts, nieces and half-siblings.

In one embodiment, the (SNP) allelic genotype is performed by hybridization-based methods or by enzyme-based methods. In one embodiment, the hybridization-based methods comprise SNP microarrays. In another embodiment, DNA sequencing is used.

### Nucleic Acid Inhibitors of FASN Expression

A powerful approach for inhibiting FASN expression is through the use of RNA interference agents. RNA interference (RNAi) uses small interfering RNA (siRNA) duplexes that target the messenger RNA encoding the target polypeptide for selective degradation, siRNA-dependent post-transcriptional silencing of gene expression involves cleaving the FASN messenger RNA molecule at a site guided by the siRNA. “RNA interference (RNAi)” is an evolutionally conserved process whereby the expression or introduction of RNA of a sequence that is identical or highly similar to a target gene results in the sequence specific degradation or specific post-transcriptional gene silencing (PTGS) of messenger RNA (mRNA) transcribed from that targeted gene (see Coburn, G. and Cullen, B. (2002) J. of Virology 76(18):9225), thereby inhibiting expression of the target gene. In one embodiment, the RNA is double stranded RNA (dsRNA). This process has been described in plants, invertebrates, and mammalian cells. In nature, RNAi is initiated by the dsRNA-specific endonuclease Dicer, which promotes processive cleavage of long dsRNA into double-stranded fragments termed siRNAs. siRNAs are incorporated into a protein complex (termed “RNA induced silencing complex,” or “RISC”) that recognizes and cleaves target mRNAs. RNAi can also be initiated by introducing nucleic acid molecules, e.g., synthetic siRNAs or RNA interfering agents, to inhibit or silence the expression of target genes.

RNAi that inhibit FASN expression are short nucleic acids and they are derived and designed to bases of FASN. The human FASN gene is located on chromosome 17, location: 17q25, 82,078,338-82,098,230 (GRCh38). Alternate gene names are SDR27X1 and OA-519. This gene encodes a multifunctional protein. Its main function is to catalyze the synthesis of palmitate from acetyl-CoA and malonyl-CoA, in the presence of NADPH, into long-chain saturated fatty acids. In some cancer cell lines, this protein has been found to be fused with estrogen receptor-alpha (ER-alpha), in which the N-terminus of FAS is fused in-frame with the C-terminus of ER-alpha. The coding transcript of FAS from this gene is NM_{—}004104.4 (SEQ. ID. No. 1) (GENBANK™).

Public access software programs and methods of predicting and selecting antisense oligonucleotides and siRNA are known in the art and are also found on the world wide web sites of GENSCRIPT™, AMBION®, DHARMACON™, OLIGOENGINE™, Wadsworth Bioinformatics Center, Whitehead Institute at the Massachusetts Institute of Technology and are also described in U.S. Pat. No. 6,060,248. After selecting the antisense oligonucleotides and siRNA sequences, these molecules can be produced biologically using an expression vector carrying the polynucleotides that encode the siRNA or antisense RNA. General molecular biological methods known in the art can be used to clone these sequences into the expression vectors. Examples of such are described herein.

In some embodiments, the siRNA ASN molecules are CCCUGAGAUCCCAGCGCUGUU (SEQ.ID.NO:2), UGGAGCGUAUCUGUGAGAA (SEQ.ID.NO:3), CCAUGGAGCGUAUCUGUGA (SEQ.ID.NO:4), UGACAUCGUCCAUUCGUUU (SEQ.ID.NO:5), GACGAGAGCACCUUUGAUG (SEQ.ID.NO:6) and GAGCGUAUCUGUGAGAAAC (SEQ.ID.NO:7). The RNA interference agent of FASN expression should not to be construed as limited to these examples. One skilled in the art can easily design other siRNAs and test their effectiveness by conventional molecular and protein methods known in the art and also described herein.

These sense and corresponding anti-sense strand oligonucleotides can be chemically synthesized, annealed and formulated for use, e.g., for direct intra-uterine injection. Alternatively, the strands can be designed into short hairpin RNA (shRNA) for plasmid- or vector-based approaches for supplying siRNAs to cells to produce stable FAS gene silencing. Examples of vectors for shRNA are #AM5779:—pSILENCER™ 4.1-CMV neo; #AM5777:—pSILENCER™ 4.1-CMV hygro; #AM5775:—pSILENCER™ 4.1-CMV puro; #AM7209:—pSILENCER™ 2.0-U6; #AM7210:—pSILENCER™ 3.0-H1; #AM5768:—pSILENCER™ 3.1-H1 puro; #AM5762:—pSILENCER™ 2.1-U6 puro; #AM5770:—pSILENCER™ 3.1-H1 neo; #AM5764:—pSILENCER™ 2.1-U6 neo; #AM5766:—pSilencer™ 3.1-H1 hygro; #AM5760:—pSILENCER™ 2.1-U6 hygro; #AM7207:—pSILENCER™ 1.0-U6 (circular) from AMBION®.

Commercial pre-designed RNA interference molecules to FAS are also available, e.g. from Santa Cruz Biotechnology, Inc., have human FAS siRNA, shRNA and lentiviral particle gene silencers: pooled siRNA molecules Fatty Acid Synthase shRNA Plasmid (m): sc-41516-SH; Fatty Acid Synthase siRNA (in): sc-41516; and Fatty Acid Synthase shRNA (m) Lentiviral Particles: sc-41516-V.

A reduction in the expression of FAS in a cell can be determined by any methods known in the art, e.g., measurement of the messenger RNA by RT-PCR or by Western blot analysis for the protein as described herein. Commercial antibodies reactive against FAS protein are widely available, e.g., from CELL SIGNALING TECHNOLOGY®, catalog #3189, ABCAM® catalog #ab22759, and Santa Cruz Biotechnology, Inc. catalog #sc-55580.

As used herein, “inhibition of FASN expression” includes any decrease in expression of the FAS protein encoded by the target gene as compared to a situation wherein no RNA interference has been induced. The decrease will be of at least 5%, 10%, 20%, 30%, 40%, 50%, 60%, 70%0, 80%, 90%, 95% or 99% or more as compared to FASN expression which has not been targeted by an RNA interfering agent.

The terms “RNA interference agent” and “RNA interference” as they are used herein are intended to encompass those forms of gene silencing mediated by double-stranded RNA, regardless of whether the RNA interfering agent comprises an siRNA, miRNA, shRNA or other double-stranded RNA molecule. “Short interfering RNA” (siRNA), also referred to herein as “small interfering RNA” is defined as an RNA agent which functions to inhibit expression of a target gene, e.g., by RNAi. An siRNA may be chemically synthesized, may be produced by in vitro transcription, or may be produced within a host cell. In one embodiment, siRNA is a double stranded RNA (dsRNA) molecule of about 15 to about 40 nucleotides in length, preferably about 15 to about 28 nucleotides, more preferably about 19 to about 25 nucleotides in length, and more preferably about 19, 20, 21, 22, or 23 nucleotides in length, and may contain a 3′ and/or 5′ overhang on each strand having a length of about 0, 1, 2, 3, 4, or 5 nucleotides. The length of the overhang is independent between the two strands, i.e., the length of the overhang on one strand is not dependent on the length of the overhang on the second strand. Preferably the siRNA is capable of promoting RNA interference through degradation or specific post-transcriptional gene silencing (PTGS) of the target messenger RNA (mRNA).

siRNAs also include small hairpin (also called stem loop) RNAs (shRNAs). In one embodiment, these shRNAs are composed of a short (e.g., about 19 to about 25 nucleotides) antisense strand, followed by a nucleotide loop of about 5 to about 9 nucleotides, and the analogous sense strand. Alternatively, the sense strand may precede the nucleotide loop structure and the antisense strand may follow. These shRNAs may be contained in plasmids, retroviruses, and lentiviruses and expressed from, for example, the pol III U6 promoter, or another promoter (see, e.g., Stewart, et al. (2003) RNA April:9(4):493-501, incorporated by reference herein in its entirety). The target gene or sequence of the RNA interfering agent may be a cellular gene or genomic sequence, e.g. the FAS sequence. An siRNA may be substantially homologous to the target gene or genomic sequence, or a fragment thereof. As used in this context, the term “homologous” is defined as being substantially identical, sufficiently complementary, or similar to the target mRNA, or a fragment thereof, to effect RNA interference of the target. In addition to native RNA molecules, RNA suitable for inhibiting or interfering with the expression of a target sequence includes RNA derivatives and analogs. Preferably, the siRNA is identical to its target. The siRNA preferably targets only one sequence. Each of the RNA interfering agents, such as siRNAs, can be screened for potential off-target effects by, for example, expression profiling. Such methods are known to one skilled in the art and are described, for example, in Jackson et al. Nature Biotechnology 6:635-637, 2003. In addition to expression profiling, one may also screen the potential target sequences for similar sequences in the sequence databases to identify potential sequences which may have off-target effects. For example, according to Jackson et al. (Id.), 15, or perhaps as few as 11 contiguous nucleotides, of sequence identity are sufficient to direct silencing of non-targeted transcripts. Therefore, one may initially screen the proposed siRNAs to avoid potential off-target silencing using the sequence identity analysis by any known sequence comparison methods, such as BLAST. siRNA sequences are chosen to maximize the uptake of the antisense (guide) strand of the siRNA into RISC and thereby maximize the ability of RISC to target human GGT mRNA for degradation. This can be accomplished by scanning for sequences that have the lowest free energy of binding at the 5′-terminus of the antisense strand. The lower free energy leads to an enhancement of the unwinding of the 5′-end of the antisense strand of the siRNA duplex, thereby ensuring that the antisense strand will be taken up by RISC and direct the sequence-specific cleavage of the human FASN mRNA. siRNA molecules need not be limited to those molecules containing only RNA, but, for example, further encompass chemically modified nucleotides and non-nucleotides, and also include molecules wherein a ribose sugar molecule is substituted for another sugar molecule or a molecule which performs a similar function. Moreover, a non-natural linkage between nucleotide residues can be used, such as a phosphorothioate linkage. The RNA strand can be derivatized with a reactive functional group of a reporter group, such as a fluorophore. Particularly useful derivatives are modified at a terminus or termini of an RNA strand, typically the 3′ terminus of the sense strand. For example, the 2′-hydroxyl at the 3′ terminus can be readily and selectively derivatized with a variety of groups. Other useful RNA derivatives incorporate nucleotides having modified carbohydrate moieties, such as 2′O-alkylated residues or 2′-O-methyl ribosyl derivatives and 2′-O-fluoro ribosyl derivatives. The RNA bases may also be modified. Any modified base useful for inhibiting or interfering with the expression of a target sequence may be used. For example, halogenated bases, such as 5-bromouracil and 5-iodouracil can be incorporated. The bases may also be alkylated, for example, 7-methylguanosine can be incorporated in place of a guanosine residue. Non-natural bases that yield successful inhibition can also be incorporated. The most preferred siRNA modifications include 2′-deoxy-2′-fluorouridine or locked nucleic acid (LAN) nucleotides and RNA duplexes containing either phosphodiester or varying numbers of phosphorothioate linkages. Such modifications are known to one skilled in the art and are described, for example, in Braasch et al., Biochemistry, 42: 7967-7975, 2003. Most of the useful modifications to the siRNA molecules can be introduced using chemistries established for antisense oligonucleotide technology. Preferably, the modifications involve minimal 2′-O-methyl modification, preferably excluding such modification. Modifications also preferably exclude modifications of the free 5′-hydroxyl groups of the siRNA. The examples herein provide specific examples of RNA interfering agents, such as shRNA molecules that effectively target FASN mRNA.

In a preferred embodiment, the RNA interference agent is delivered or administered in a pharmaceutically acceptable carrier. Additional carrier agents, such as liposomes, can be added to the pharmaceutically acceptable carrier. In another embodiment, the RNA interference agent is delivered by a vector encoding small hairpin RNA (shRNA) in a pharmaceutically acceptable carrier to the cells in an organ of an individual. The shRNA is converted by the cells after transcription into siRNA capable of targeting the FASN mRNA.

In one embodiment, the vector for delivering the shRNA is a regulatable vector, such as a tetracycline inducible vector. Methods described, for example, in Wang et al. Proc. Natl. Acad. Sci. 100: 5103-5106, using pTet-On vectors (BD Biosciences Clontech, Palo Alto, Calif.) can be used. In one embodiment, the RNA interference agents used in the methods described herein are taken up actively by cells in vivo following intravenous injection or release from a suppository at the vagina, cervix or uterus, without the use of a vector, illustrating efficient in vivo delivery of the RNA interfering agents. One method to deliver the siRNAs is catheterization of the blood supply vessel of the uterus. Other strategies for delivery of the RNA interference agents, e.g., the siRNAs or shRNAs used in the methods described herein, may also be employed, such as, for example, delivery by a vector, e.g., a plasmid or viral vector, e.g., a lentiviral vector. Such vectors can be used as described, for example, in Xiao-Feng Qin et al. Proc. Natl. Acad. Sci. U.S.A., 100: 183-188. Other delivery methods include delivery of the RNA interfering agents, e.g., using a basic peptide by conjugating or mixing the RNA interfering agent with a basic peptide, e.g., a fragment of a TAT peptide, mixing with cationic lipids or formulating into particles. The RNA interference agents, e.g., the siRNAs targeting FASN mRNA, may be delivered singly, or in combination with other RNA interference agents, e.g., siRNAs, such as, for example siRNAs directed to other cellular genes. FAS siRNAs may also be administered in combination with other pharmaceutical agents which are used to treat or prevent diseases or disorders associated with oxidative stress, especially respiratory diseases, and more especially asthma. Synthetic siRNA molecules, including shRNA molecules, can be obtained using a number of techniques known to those of skill in the art. For example, the siRNA molecule can be chemically synthesized or recombinantly produced using methods known in the art, such as using appropriately protected ribonucleoside phosphoramidites and a conventional DNA/RNA synthesizer (see, e.g., Elbashir, S. M. et al. (2001) Nature 411:494-498; Elbashir, S. M., W. Lendeckel and T. Tuschl (2001) Genes & Development 15:188-200; Harborth, J. et al. (2001) J. Cell Science 114:4557-4565; Masters, J. R. et al. (2001) Proc. Natl. Acad. Sci., USA 98:8012-8017; and Tuschl, T. et al. (1999) Genes & Development 13:3191-3197). Alternatively, several commercial RNA synthesis suppliers are available including, but not limited to, Proligo (Hamburg, Germany), Dharmacon Research (Lafayette, Colo., USA), Pierce Chemical (part of Perbio Science, Rockford, Ill., USA), Glen Research (Sterling, Va., USA), ChemGenes (Ashland, Mass., USA), and Cruachem (Glasgow, UK). As such, siRNA molecules are not overly difficult to synthesize and are readily provided in a quality suitable for RNAi. In addition, dsRNAs can be expressed as stem loop structures encoded by plasmid vectors, retroviruses and lentiviruses (Paddison, P. J. et al. (2002) Genes Dev. 16:948-958; McManus, M. T. et al. (2002) RNA 8:842-850; Paul, C. P. et al. (2002) Nat. Biotechnol. 20:505-508; Miyagishi, M. et al. (2002) Nat. Biotechnol. 20:497-500; Sui, G. et al. (2002) Proc. Natl. Acad. Sci., USA 99:5515-5520; Brummelkamp, T. et al. (2002) Cancer Cell 2:243; Lee, N. S., et al. (2002) Nat. Biotechnol. 20:500-505; Yu, J. Y., et al. (2002) Proc. Natl. Acad. Sci., USA 99:6047-6052; Zeng, Y., et al. (2002) Mol. Cell 9:1327-1333; Rubinson, D. A., et al. (2003) Nat. Genet. 33:401-406; Stewart, S. A., et al. (2003) RNA 9:493-501). These vectors generally have a pol III promoter upstream of the dsRNA and can express sense and antisense RNA strands separately and/or as a hairpin structures. Within cells, Dicer processes the short hairpin RNA (shRNA) into effective siRNA. The targeted region of the siRNA molecule for inhibiting FASN expression can be selected from a given target gene sequence, e.g., a FAS coding sequence, beginning from about 25 to 50 nucleotides, from about 50 to 75 nucleotides, or from about 75 to 100 nucleotides downstream of the start codon. Nucleotide sequences may contain 5′ or 3′ UTRs and regions nearby the start codon. One method of designing an siRNA molecule for inhibiting FASN expression involves identifying the 23 nucleotide sequence motif AA(N19)TT (SEQ. ID. NO. 8) (where N can be any nucleotide) and selecting hits with at least 25%, 30%, 35%, 40%, 45%, 50%, 55%, 60%, 65%, 70% or 75% G/C content. The “TT” portion of the sequence is optional. Alternatively, if no such sequence is found, the search may be extended using the motif NA(N21), where N can be any nucleotide. In this situation, the 3′ end of the sense siRNA may be converted to TT to allow for the generation of a symmetric duplex with respect to the sequence composition of the sense and antisense 3′ overhangs. The antisense siRNA molecule may then be synthesized as the complement to nucleotide positions 1 to 21 of the 23 nucleotide sequence motif. The use of symmetric 3′ TT overhangs may be advantageous to ensure that the small interfering ribonucleoprotein particles (siRNPs) are formed with approximately equal ratios of sense and antisense target RNA-cleaving siRNPs (Elbashir et al., (2001) supra and Elbashir et al., 2001 supra). Analysis of sequence databases, including but not limited to the NCBI, BLAST, and GenSeq as well as commercially available oligosynthesis companies such as OLIGOENGINE®, may also be used to select siRNA sequences against EST libraries to ensure that only one gene is targeted.

### Delivery of RNA Interfering Agents

Methods of delivering RNA interference agents, e.g., an siRNA, or vectors containing an RNA interference agent, to the target cells in the uterine wall for uptake include injection of a composition containing the RNA interference agent, e.g., an siRNA, or directly contacting the uterine wall cells with a composition comprising an RNA interference agent, e.g., an siRNA via an inserted suppository. In another embodiment, RNA interference agent, e.g., an siRNA may be injected directly into any blood vessel, such as vein, artery, venule or arteriole, via, e.g., hydrodynamic injection or catheterization. Administration may be by a single injection or by two or more injections. The RNA interference agent is delivered in a pharmaceutically acceptable carrier. One or more RNA interference agents may be used simultaneously. In one embodiment, only one RNA interference agent e.g. siRNA, that targets the human FASN is used. In one embodiment, a pool of RNA interference agents that targets the human tASN is used. In one embodiment, specific cells are targeted with RNA interference, limiting potential side effects of RNA interference caused by non-specific targeting of RNA interference. The method can use, for example, a complex or a fusion molecule comprising a cell targeting moiety and an RNA interference binding moiety that is used to deliver RNA interference effectively into cells. For example, an antibody-protamine fusion protein when mixed with siRNA, binds siRNA and selectively delivers the siRNA into cells expressing an antigen recognized by the antibody, resulting in silencing of gene expression only in those cells that express the antigen. The siRNA or RNA interference-inducing molecule binding moiety is a protein or a nucleic acid binding domain or fragment of a protein, and the binding moiety is fused to a portion of the targeting moiety. The location of the targeting moiety can be either in the carboxyl-terminal or amino-terminal end of the construct or in the middle of the fusion protein. For example, the uterine cells respond to estrogen hormones. The targeting moiety can be that for the estrogen hormone receptors.

A viral-mediated delivery mechanism can also be employed to deliver siRNAs to cells in vitro and in vivo as described in Xia, H. et al. (2002) Nat Biotechnol 20(10):1006). Plasmid- or viral-mediated delivery mechanisms of shRNA may also be employed to deliver shRNAs to cells in vitro and in viva as described in Rubinson, D. A., et al. ((2003) Nat. Genet. 33:401-406) and Stewart, S. A., et al. ((2003) RNA 9:493-501). The RNA interference agents, e.g., the siRNAs or shRNAs, can be introduced along with components that perform one or more of the following activities: enhance uptake of the RNA interfering agents, e.g., siRNA, by the cell, e.g., lymphocytes or other cells, inhibit annealing of single strands, stabilize single strands, or otherwise facilitate delivery to the target cell and increase inhibition of the target gene, e.g., FASN. The dose of the particular RNA interfering agent will be in an amount necessary to effect RNA interference, e.g., post translational gene silencing (PTGS), of the particular target gene, thereby leading to inhibition of target gene expression or inhibition of activity or level of the protein encoded by the target gene FASN.

In some embodiments, the siRNA, dsRNA, or shRNA vector directed against FASN is formulated for administration systemically, such as intravenously, e.g. via central venous catheter (CVC or central venous line or central venous access catheter) placed into a large vein in the neck (internal jugular vein), chest (subclavian vein) or groin (femoral vein). Methods of systemic delivery of siRNA, dsRNA, or shRNA vector are well known in the art, e.g. as described herein and in Mammoto T., et al, 2007 (J. Biol. Chem., 282:23910-23918), Gao and Huang, 2008, (Mol. Pharmaceutics, Web publication December 30) and a review by Rossil, 2006, Gene Therapy, 13:583-584. Alternatively, the siRNA, dsRNA, or shRNA vector can be delivered via catheter, by means of a uterine, vaginal or cervical suppository, or by means of an implant thereon. The siRNA, dsRNA, or shRNA vector can be formulated in various ways, e.g., conjugation of a cholesterol moiety to one of the strands of the siRNA duplex, conjugated liposomes and micelles (Soutschek J. et. al. 2004, Nature, 432:173-178), complexing of siRNAs to protamine fused with an antibody fragment for receptor-mediated targeting of siRNAs (Song E, et al. 2005, Nat. Biotechnol., 23: 709-717) and the use of a lipid bilayer system by Morrissey et al. 2005 (Nat. Biotechnol., 23: 1002-1007). The lipid bilayer system produces biopolymers that are in the 120 nanometer diameter size range, and are labeled as SNALPs, for Stable-Nucleic-Acid-Lipid-Particles. The lipid combination protects the siRNAs from serum nucleases and allows cellular endosomal uptake and subsequent cytoplasmic release of the siRNAs (see WO/2006/007712). These references are incorporated by reference in their entirety.

### Formulation and Administration

In one embodiment, the FAS inhibitor is delivered in a pharmaceutically acceptable carrier.

In one embodiment, the term “pharmaceutically acceptable” means approved by a regulatory agency of the Federal or a state government or listed in the U.S. Pharmacopeia or other generally recognized pharmacopeia for use in animals, and more particularly in humans. Specifically, it refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsions, tablets, pills, capsules, powders, sustained-release formulations, and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in Remington's Pharmaceutical Sciences, 18th Ed., Gennaro, ed. (Mack Publishing Co., 1990). The formulation should suit the mode of administration. Additional carrier agents, such as liposomes, can be added to the pharmaceutically acceptable carrier.

As used herein, “administering” refers to the placement of an inhibitor of FAS into a subject by a method or route which results in at least partial localization of the inhibitor at a desired site. The FAS inhibitor e.g., a small molecule FAS inhibitor or shRNA, which inhibits FAS can be administered by any appropriate route which results in effective treatment in the subject, i.e. administration results in delivery to a desired location in the subject where at least a portion of the composition delivered, i.e. at least one agent which inhibits FAS activity or tA-VSN expression, is active in the desired site for a period of time. The period of time the FAS inhibitor is active depends on the half-life in vivo after administration to a subject, and can be as short as a few hours, e.g. twenty-four hours, to a few days, to as long as several years. Modes of administration include injection, infusion, instillation, vaginal suppository, cervical suppository, percutaneous implantation or ingestion. “Injection” includes, without limitation, intravenous, intramuscular, intraarterial, intraventricular, intradermal, intraperitoneal, subcutaneous, subcuticular injection and infusion.

As used herein, the term “comprising” or “comprises” is used in reference to methods, and respective component(s) thereof, that are essential to the claims, yet open to the inclusion of unspecified elements, whether essential or not. The use of“comprising” indicates inclusion rather than limitation.

The term “consisting of” refers to methods, and respective components thereof as described herein, which are exclusive of any element not recited in that description of the embodiment.

Therapeutic compositions contain a physiologically tolerable carrier together with at least an inhibitor of FAS as described herein, dissolved or dispersed therein as an active ingredient. In one embodiment, the therapeutic composition is not immunogenic when administered to a mammal or human patient for therapeutic purposes. As used herein, the terms “pharmaceutically acceptable”, “physiologically tolerable” and granunatical variations thereof, as they refer to compositions, carriers, diluents and reagents, are used interchangeably and represent that the materials are capable of administration to or upon a mammal without the production of undesirable physiological effects such as nausea, dizziness, gastric upset and the like. A pharmaceutically acceptable carrier will not promote the raising of an immune response to an agent with which it is admixed, unless so desired. The preparation of a pharmacological composition that contains active ingredients dissolved or dispersed therein is well understood in the art and need not be limited based on formulation. Compositions can be prepared as injectable either as liquid solutions or suspensions, however, solid forms suitable for solution, or suspensions; in liquid prior to use can also be prepared. The preparation can also be emulsified or presented as a liposome composition. The FAS inhibitors can also be conjugated with lipids. e.g., amphipathic lipids, for stability and delivery purposes. The conjugation bonds are reversible and are broken or dissolved when the FAS inhibitors are delivered to the target destination(s). Alternatively, the FAS inhibitors described herein are prepared as a solid or semi-solid or emulsion in suppository. The suppository is inserted as a solid into the uterus, vagina or cervix, and will dissolve or melt inside the body to deliver the FAS inhibitors received. Liquid suppository comprising the FAS inhibitors described herein can be injected with a syringe, into the uterus. The FAS inhibitors described herein can be mixed with excipients which are pharmaceutically acceptable and compatible with the active ingredient and in amounts suitable for use in the therapeutic methods described herein. Specifically contemplated pharmaceutical compositions are FAS inhibitors (e.g., small molecule inhibitors or active RNAi ingredients) in a preparation for delivery as described herein above, or in references cited and incorporated herein in that section. Suitable excipients include, for example, water, saline, dextrose, glycerol, ethanol or the like and combinations thereof. In addition, if desired, the composition can contain minor amounts of auxiliary substances such as wetting or emulsifying agents, pH buffering agents and the like which enhance the effectiveness of the active ingredient. The therapeutic composition comprising the FAS inhibitors described herein can include pharmaceutically acceptable salts of the components therein. Pharmaceutically acceptable salts include the acid addition salts (formed with the free amino groups of the polypeptide) that are formed with inorganic acids such as, for example, hydrochloric or phosphoric acids, or such organic acids as acetic, tartaric, mandelic and the like. Salts formed with the free carboxyl groups can also be derived from inorganic bases such as, for example, sodium, potassium, ammonium, calcium or ferric hydroxides, and such organic bases as isopropylamine, trimethylamine, 2-ethylamino ethanol, histidine, procaine and the like. Physiologically tolerable carriers are well known in the art. Exemplary liquid carriers are sterile aqueous solutions that contain no materials in addition to the active ingredients and water, or contain a buffer such as sodium phosphate at physiological pH value, physiological saline or both, such as phosphate-buffered saline. Still further, aqueous carriers can contain more than one buffer salt, as well as salts such as sodium and potassium chlorides, dextrose, polyethylene glycol and other solutes. Liquid compositions can also contain liquid phases in addition to and to the exclusion of water. Exemplary of such additional liquid phases are glycerin, vegetable oils such as cottonseed oil, and water-oil emulsions. The amount of FAS inhibitors used in the methods described herein that will be effective in the treatment of a particular disorder or condition will depend on the nature of the disorder or condition, and can be determined by standard clinical techniques.

Routes of administration include, but are not limited to, direct injection, intradermal, intramuscular, intraperitoneal, intravenous, subcutaneous, intrauterine and oral routes. The FAS inhibitors can be administered by any convenient route, for example by infusion, suppository or bolus injection, by absorption through epithelial or mucocutaneous linings (e.g., oral mucosa, rectal and intestinal mucosa, etc.) and may be administered together with other biologically active agents. Administration can be systemic or local.

The precise dose and formulation to be employed depend upon the potency of the inhibitor, and include amounts large enough to produce the desired effect, e.g., a reduction in size and/or growth of the UL in, on, or in the surrounding peritoneum of the uterus. The dosage should not be so large as to cause unacceptable adverse side effects. Generally, the dosage will vary with the type of FAS inhibitor (e.g., polyphenol, small molecule, dietary compound, siRNA, etc.), and with the age, condition, and consideration of the size of the tumors in the subject. Dosage and formulation of the FAS inhibitor will also depend on the route of administration, and the mass and number of the UL in the uterus, and should be decided according to the judgment of the practitioner and each subject's circumstances. Effective doses can be extrapolated from dose-response curves derived from in vilro or animal model test systems.

The dosage can be determined by one of skill in the art and can also be adjusted by the individual physician in the event of any complication. Typically, the dosage ranges from 0.001 mg/kg body weight to 5 g/kg body weight. In some embodiments, the dosage range is from 0.001 mg/kg body weight to 1 g/kg body weight, from 0.001 mg/kg body weight to 0.5 g/kg body weight, from 0.001 mg/kg body weight to 0.1 g/kg body weight, from 0.001 mg/kg body weight to 50 mg/kg body weight, from 0.001 mg/kg body weight to 25 mg/kg body weight, from 0.001 mg/kg body weight to 10 mg/kg body weight, from 0.001 mg/kg body weight to 5 mg/kg body weight, from 0.001 mg/kg body weight to 1 mg/kg body weight, from 0.001 mg/kg body weight to 0.1 mg/kg body weight, from 0.001 mg/kg body weight to 0.005 mg/kg body weight. Alternatively, in some embodiments the dosage range is from 0.1 g/kg body weight to 5 g/kg body weight, from 0.5 g/kg body weight to 5 g/kg body weight, from 1 g/kg body weight to 5 g/kg body weight, from 1.5 g/kg body weight to 5 g/kg body weight, from 2 g/kg body weight to 5 g/kg body weight, from 2.5 g/kg body weight to 5 g/kg body weight, from 3 g/kg body weight to 5 g/kg body weight, from 3.5 g/kg body weight to 5 g/kg body weight, from 4 g/kg body weight to 5 g/kg body weight, from 4.5 g/kg body weight to 5 g/kg body weight, from 4.8 g/kg body weight to 5 g/kg body weight. In one embodiment, the dose range is from 5 g/kg body weight to 30 g/kg body weight. Alternatively, the dose range will be titrated to maintain serum levels between 5 g/mL and 30 g/mL.

Administration of the doses recited above can be repeated for a limited period of time. In some embodiments, the doses are given once a day, or multiple times a day, for example but not limited to three times a day. In one embodiment, the doses recited above are administered daily for several weeks or months. The duration of treatment depends upon the female subject's clinical progress and responsiveness to therapy, e.g., shrinkage of UL tumor sizes. Continuous, relatively low maintenance doses are contemplated after an initial higher therapeutic dose. As exemplary, the FAS inhibitor and a pharmaceutically acceptable carrier can be formulated for direct application by suppository adjacent to the UL in the uterus.

In one embodiment, the FAS inhibitor is an RNA interference molecule such as an siRNA. Such siRNA is delivered by delivering a vector encoding small hairpin RNA (shRNA) in a pharmaceutically acceptable carrier to the cells in the uterus of a female subject having UL. The shRNA is converted by the cells after transcription into siRNA capable of targeting FAS. In one embodiment, the vector can be a regulatable vector, such as tetracycline inducible vector. Such vectors with inducible promoters are well known in the art and are also easily found in the commercial sector, e.g. pSingle-tTS-shRNA vector from CLONTECH®.

Efficacy testing can be performed during the course of treatment using the methods described herein, e.g., ultrasound, MRI and CT to monitor the shrinkage in size of the UL in the treated subject. A decrease in size of the tumors during and after treatment indicates that the treatment is effective in reducing UL. Measurements of the degree of severity of a number of symptoms associated with UL are also noted prior to the start of a treatment and then at a later specific time period after the start of the treatment. For example, the severity of menstrual bleeding is scored prior to and during treatment. The severity of menstrual bleeding after the treatment is compared to those before the treatment. A decrease in the menstrual bleeding after treatment indicates that the treatment is effective in reducing UL. A skilled physician will be able to ascertain the UL sizes and related symptoms by known methods in the art and those described herein.

The references cited herein and throughout the specification are incorporated herein by reference.

The present claims are defined in any of the following alphabetized paragraphs:
[A] The A fatty acid synthase (FAS) inhibitor for use in the treatment of uterine leiomyomata (UL).
[B] The FAS inhibitor of paragraph [A], wherein the FAS inhibitor inhibits FAS activity.
[C] The FAS inhibitor of paragraph [A] or [B], wherein the FAS inhibitor is selected from the group consisting of a small molecule inhibitor, a polyphenol and a dietary compound.
[D] The FAS inhibitor of paragraph [C], wherein the inhibitor of FAS activity is selected from the group consisting of (S)—((S)-1-((2S,3S)-3-hexyl-4-oxooxetan-2-yl)tridecan-2-yl) 2-formamido-4-methylpentanoate, 3-Carboxy-4-octyl-2-methylenebutyrolactone, trans-4-Carboxy-5-octyl-3-methylenebutyrolactone (C75), cerulenin. C93 (FAS93), FAS31, C247, GSK837149A, platensimycin, 3-aryl-4-hydroxyquinolin-2(1H)-one scaffold (MERCK), and bisamide scaffold (AstraZeneca).
[E] The FAS inhibitor of paragraph [C], wherein the inhibitor of FAS activity is selected from the group consisting of epigallocatechin, luteolin, taxifolin, kaempferol, quercetin and apigenin.
[F] The FAS inhibitor of paragraph [C], wherein the inhibitor of FAS activity is selected from the group consisting of catechin, soy protein and monounsaturated fatty acid oleic acid (18:1, n-9).
[G] The FAS inhibitor of paragraph [C], wherein the inhibitor of FAS activity is selected from the group consisting of polyhydroxylated compounds of US 2010/0190856, cerulenin compounds of U.S. Pat. No. 5,981,575, spirocyclic piperidines of WO2012/064642, and compounds of US 20100022630.
[H] The FAS inhibitor of paragraph [A], wherein the FAS inhibitor inhibits FAS expression.
[I] The FAS inhibitor of paragraph [H], wherein the inhibitor of FAS expression is selected from a small molecule and a nucleic acid.
[J] The FAS inhibitor of paragraph [H] or [I], wherein the FAS inhibitor is a FAS specific RNA interference agent, or a vector encoding a FAS specific RNA interference agent.
[K] The FAS inhibitor of paragraph [J], wherein the RNA interference agent is a double stranded RNA (dsRNA).
[L] The FAS inhibitor of paragraph [J] or [K], wherein the RNA interference agent comprises one or more of the nucleotide sequences of SEQ. ID. NOS: 2-7.
[M] The FAS inhibitor of any one of paragraphs [A]-[L], wherein the FAS inhibitor is formulated for administration by injection, infusion, instillation, vaginal suppository, cervical suppository, percutaneous implantation, or ingestion.
[N] Use of a fatty acid synthase (FAS) inhibitor in the manufacture of a medicament for the treatment of uterine leiomyomata (UL).
[O] The use of paragraph [N], wherein the FAS inhibitor inhibits FAS activity.
[P] The use of paragraph [N] or [O], wherein the FAS inhibitor is selected from the group consisting of a small molecule inhibitor, a polyphenol and a dietary compound.
[Q] The use of paragraph [P], wherein the inhibitor of FAS activity is selected from the group consisting of (S)—((S)-1-((2S,3S)-3-hexyl-4-oxooxetan-2-yl)tridecan-2-yl) 2-formamido-4-methylpentanoate, 3-Carboxy-4-octyl-2-methylenebutyrolactone, trans-4-Carboxy-5-octyl-3-methylenebutyrolactone (C75), cerulenin, C93 (FAS93), FAS31, C247, GSK837149A, platensimycin, 3-aryl-4-hydroxyquinolin-2(l H)-one scaffold (MERCK), and bisamide scaffold (AstraZeneca).
[R] The use of paragraph [P], wherein the inhibitor of FAS activity is selected from the group consisting of epigallocatechin, luteolin, taxifolin, kaempferol, quercetin and apigenin.
[S] The use of paragraph [P], wherein the inhibitor of FAS activity is selected from the group consisting of catechin, soy protein and monounsaturated fatty acid oleic acid (18:1, n-9).
[T] The use of paragraph [P], wherein the inhibitor of FAS activity is selected from the group consisting of polyhydroxylated compounds of US 2010/0190856, cerulenin compounds of U.S. Pat. No. 5,981,575, spirocyclic piperidines of WO2012/064642, and compounds of US 20100022630.
[U] The use of paragraph [N], wherein the FAS inhibitor inhibits FAS expression.
[V] The use of paragraph [U], wherein the inhibitor of FAS expression is selected from a small molecule and a nucleic acid.
[W] The use of paragraph [U] or [V], wherein the FAS inhibitor is a FAS specific RNA interference agent, or a vector encoding a FAS specific RNA interference agent.
[X] The use of paragraph [W], wherein the RNA interference agent is a double stranded RNA (dsRNA).
[Y] The use of paragraph [W] or [X], wherein the RNA interference agent comprises one or more of the nucleotide sequences of SEQ. ID. NOS: 2-7.
[Z] The use of any one of paragraphs [N]-[Y], wherein the FAS inhibitor is formulated for administration by injection, infusion, instillation, vaginal suppository, cervical suppository, percutaneous implantation, or ingestion.
[AA] A composition for the treatment of uterine leiomyomata (UL) comprising an inhibitor of a fatty acid synthase (FAS) of any one of claims [A]-[M] and a pharmaceutical acceptable carrier.
[BB] A method of treatment of uterine leiomyomata (UL) in a female subject in need thereof comprising administering a therapeutically effective amount of fatty acid synthase (FAS) inhibitor of any one of claims [A]-[M] or a composition comprising an inhibitor of a fatty acid synthase (FAS) of claim [AA].
[CC] A method of treatment of uterine leiomyomata (UL) in a female subject in need thereof comprising administering a composition comprising an inhibitor of a fatty acid synthase (FAS) and a pharmaceutical acceptable carrier.
[DD] The method of paragraph [CC], wherein the composition comprising an inhibitor of FAS inhibits FAS activity.
[EE] The method of paragraph [CC] or [DD], wherein the inhibitor of FAS activity is selected from the group consisting of a small molecule inhibitor, a polyphenol and a dietary compound.
[FF] The method of paragraph [EE], wherein the inhibitor of FAS activity is selected from the group consisting of (S)—((S)-1-((2S,3S)-3-hexyl-4-oxooxetan-2-yl)tridecan-2-yl) 2-formamido-4-methylpentanoate, 3-Carboxy-4-octyl-2-methylenebutyrolactone, trans-4-Carboxy-5-octyl-3-methylenebutyrolactone (C75), cerulenin, C93 (FAS93), FAS31, C247, GSK837149A, platensimycin, 3-aryl-4-hydroxyquinolin-2(1H)-one scaffold (MERCK), and bisamide scaffold (AstraZeneca).
[GG] The method of paragraph [EE], wherein the inhibitor of FAS activity is selected from the group consisting of epigallocatechin, luteolin, taxifolin, kaempferol, quercetin and apigenin.
[HH] The method of paragraph [EE], wherein the inhibitor of FAS activity is selected from the group consisting of catechin, soy protein and monounsaturated fatty acid oleic acid (18:1, n-9).
[II] The method of paragraph [EE], wherein the inhibitor of FAS activity is selected from the group consisting of polyhydroxylated compounds of US 2010/0190856, cerulenin compounds of U.S. Pat. No. 5,981,575, spirocyclic piperidines of WO2012/064642, and compounds of US 20100022630.
[JJ] The method of paragraph [CC], wherein the composition comprising an inhibitor of FAS inhibits FAS expression.
[KK] The method of paragraph [JJ], wherein the inhibitor of FAS expression is selected from a small molecule and a nucleic acid.
[LL] The method of paragraph [JJ] or [KK], wherein the inhibitor of FAS expression is a FAS specific RNA interference agent, or a vector encoding a FAS specific RNA interference agent.
[MM] The method of paragraph [LL], wherein said RNA interference agent is a double stranded RNA (dsRNA).
[NN] The method of paragraph [LL] or [MM], wherein said RNA interference agent comprises one or more of the nucleotide sequences of SEQ. ID. NOS: 2-7.
[OO] The method of any one of paragraphs [CC]-[MM], wherein the composition is formulated for administration by injection, infusion, instillation, vaginal suppository, cervical suppository, percutaneous implantation or ingestion.
[PP] The method of any one of paragraphs [CC]-[EE], wherein the female subject has been diagnosed with uterine leiomyomata.
[QQ] The method of any one of paragraphs [CC]-[PP], wherein the female subject has the minor SNP allele, adenine (A) at rs4247357 at chromosome 17.
[RR] The method of any one of paragraphs [CC]-[QQ], wherein the female subject is homozygous for the minor (A) SNP allele at rs4247357 at chromosome 17.
[SS] The method of any one of paragraphs [CC]-[QQ], wherein the female subject is heterozygous for the minor (A) SNP allele at rs4247357 at chromosome 17.
[TT] A method comprising:
   providing a genetic sample from a female subject for assessing the increased likelihood of developing uterine leiomyomata (UL);
   determining the single nucleotide polymorphism (SNP) allelic genotype at rs4247357 at chromosome 17,
   wherein a homozygosity (AA) and heterozygosity (AC) for the minor SNP allele indicates an increased likelihood of developing UL.
[UU] The method of paragraph [TT], wherein the genetic sample from a female subject is a tissue sample comprising deoxyribonucleic acid (DNA).
[VV] The method of paragraph [TT] or [UU], wherein the genetic sample from a female subject is selected from the group consisting of a blood sample, a saliva sample, a skin sample, a hair bulb and an epithelial sample.
[WW] The method of any one of paragraphs [TT]-[VV], wherein the female subject has reached puberty.
[XX] The method of any one of paragraphs [TT]-[WW], wherein the female subject has not entered perimenopause or menopause.
[YY] The method of any one of paragraphs [TT]-[WW], wherein the female subject has entered perimenopause or menopause.
[ZZ] The method of claim any one of paragraphs [TT]-[YY], wherein the female subject is on hormone replacement therapy.
[AAA] The method of any one of paragraphs [TT]-[ZZ], wherein the female subject is between the ages of 7-70.
[BBB] The method of any one of paragraphs [TT]-[VV], wherein the female subject has not reached menarche.
[CCC] The method of any one of paragraphs [TT]-[BBB], wherein the female subject has at least one first and/or second degree relative who has or has had UL.
[DDD] The method of any one of paragraphs [TT]-[CCC], wherein the (SNP) allelic genotype is performed by hybridization-based methods or by enzyme-based methods.
[EEE] The method of paragraph [DDD], wherein the hybridization-based methods comprise SNP microarrays.

This disclosure is further illustrated by the following example which should not be construed as limiting. The contents of all references cited throughout this application, as well as the figures and table are incorporated herein by reference.

Those skilled in the art will recognize, or be able to ascertain using not more than routine experimentation, many equivalents to the specific embodiments described herein. Such equivalents are intended to be encompassed by the following claims.

### Example

### Materials and Methods

### Finding Genes for Fibroids Linkage Analysis

Sister pairs affected with UL were recruited for the “Finding Genes for Fibroids” (FGFF) study. Approximately 385 sister pairs were consented for this project. Both sisters have medical record confirmed UL, provide a blood sample and complete a questionnaire on clinical, reproductive, sexual, and family history relating to UL. Other family members of the sisters also contributed samples and completed questionnaires. Study participants were recruited under an IRB protocol approved by the Partners HealthCare System Human Research Committee. DNA was isolated using a PUREGENE Blood Kit (Gentra, Minneapolis, Minn.) and the DNA, pedigree information, and UL affection status were provided to the genotyping core at The Center for Inherited Disease Research (CIDR) at Johns Hopkins University. A whole genome SNP linkage scan was performed using ILLUMINA's Human Linkage-12 Genotyping BeadChip (San Diego, Calif.). Two families with multiple Mendelian inconsistencies were excluded in addition to 14 SNP markers due to low quality genotype calls.

Linkage analysis was performed with GENEHUNTER software using samples from self-reported white sister pairs and family members, which comprised 261 families with a total of 1103 individuals. The minor allele frequency (MAF) of each SNP was calculated using the genotype information from one sister in each family. SNPs were pruned using PLINK software (16) based on an r² of 0.2 resulting in a total of 4,196 SNPs for the final analysis. Sib-pair analysis was carried out with UL status as the phenotype and significant linkage with UL was defined as a LOD score greater than 3.6. Despite particular interest in recruitment of black women, the FGFF study has not yet reached an appropriate number of sister pairs for this important analysis.

### Women's Genome Health Study Association Study

The Women's Genome Health Study (WGHS) (17) is a prospective cohort of female North American health care professionals representing participants in the Women's Health Study (WHS) who provided a blood sample at baseline and consent for blood-based analyses. Participants in the WHS were 45 years of age or older at enrollment and free of cardiovascular disease, cancer or other major chronic illness. Additional information related to health and lifestyle were collected by questionnaires throughout the WHS trial and continuing observational follow-up. WHS participants were asked if they had ever been diagnosed with UL, their age at diagnosis, whether their mother or sister had ever been diagnosed with UL and their history of hysterectomy.

Genotyping in the WGHS sample was performed using the HumanHap300 Duo “+” chips or the combination of the HumanHap300 Duo and ISELECT chips (ILLUMINA, San Diego, Calif.) with the INFINIUM II protocol. In either case, the custom SNP content was the same; these custom SNPs were chosen without regard to minor allele frequency (MAF) to saturate candidate genes for cardiovascular disease as well as to increase coverage of SNPs with known or suspected biological function, e.g. disease association, non-synonymous changes, substitutions at splice sites, etc. For quality control, all samples were required to have successful genotyping using the BEADSTUDIO v. 3.3 software (ILLUMINA) for at least 98% of the SNPs. A subset of 23,294 individuals were identified with self-reported European ancestry, verified on the basis of multidimensional scaling analysis of identity by state using 1,443 ancestry informative markers in PLINK v. 1.06 (16). The final dataset of these individuals included SNPs with MAF>1%, successful genotyping in 90% of subjects, and deviations from Hardy-Weinberg equilibrium not exceeding p=10. Among the final 23,294 individuals of verified European ancestry, genotypes for a total of 2,608,509 SNPs were imputed from the experimental genotypes and linkage disequilibrium (LD) relationships implicit in the HapMap r. 22 CEU samples. Imputed SNPs were used to define the region of LD surrounding association signals found with genotyped SNPs.

UL status, age at diagnosis, mother or sister UL status, and history of hysterectomy were ascertained by recall in the 2009 WGHS questionnaire. UL cases and controls from WGHS were stratified based on these four variables in an attempt to identify women most and least likely to have a genetic basis for UL. Any participant who answered “not sure” for UL status or mother/sister UL status was excluded from the analysis. Participants were also excluded who reported an age of UL diagnosis at less than 20 years or at greater than 70 years. Cases included women who answered “yes” for UL status, “yes” for their mother or sister UL status, and either had an age of diagnosis under 40 years or had a hysterectomy. Controls included women who answered “no” to UL status, mother/sister UL status and who had not had a hysterectomy. Women not qualifying for either of these groups were excluded from the analysis. Following stratification there were 746 cases and 4,487 controls. Association analysis was performed on the set of 339,187 genotyped SNPs in PLINK using the standard case/control test and p-values less than 5×10⁻⁸ were considered significant.

### Australian Cohort Association Study

Individuals comprising the cohort from the Queensland Institute of Medical Research (QIMR) were women who had been consented and genotyped previously on ILLUMINA's 317K, 370K or 610K SNP platforms as pan of a larger collection of genome-wide association studies conducted at QIMR(18; 19). Case samples (n=484) were selected from amongst women originally recruited into a study of genetic factors underlying endometriosis (20) and a twin study of gynecological health (21). For both studies, women had completed questionnaires on various aspects of reproductive health, and cases had answered ‘yes’ to the ‘uterine fibroids’ option of the question “Have you ever had any of the following conditions?” Controls (n=610) were taken from amongst twin pairs from the gynecological health study where both sisters had answered ‘no’ to the question on uterine fibroids (one sample per twin pair). A standard case/control association analysis was performed on the set of 269,629 SNPs genotyped in common between all samples and passing all QC metrics in PLINK (16). Approval for the studies was granted by the Human Research Ethics Committee at QIMR and the Australian Twin Registry. All gene annotations and base pair positions are derived from the human genome sequence hg 18 (NCBI build 36.1).

### Demographics

Demographics of the FGFF, WGHS and Australian cohorts were considered, focusing on variables most relevant to UL diagnosis. In the WGHS and Australian cohorts, cases have a slightly higher BMI and have decreased stature compared to controls although the difference is not significant in the WGHS population (defined as a P>0.05) (Table 2). Previous studies have also reported a correlation with UL diagnosis and a higher BMI (22; 23). WGHS and Australian cases also have a younger age at menarche which could be expected as UL are hormone dependent neoplasms and a younger age at menarche is associated with longer reproductive years and additional years of hormone exposure. Lastly, although Australian cases were not selected based on a history of hysterectomy, UL cases have a very significant increase in hysterectomy prevalence reflecting the fact that UL are the leading cause for this surgery. Overall, demographics of the WGHS and Australian cohorts were unremarkable and confirmed previously reported UL associations.

### FAS Immunohistochemistry

Fatty acid synthase (FAS) protein levels in UL and myometrium tissue were analyzed by immunohistochemistry of tissue microarrays (TMAs) consisting of tissue sections from 200 women, 36 myometrium samples and 337 UL samples. The TMA includes matched paraffin embedded formalin fixed myometrium and UL samples from 33 women. Two TMAs were constructed using sections from different areas of the same tissue. DNA corresponding to 106 tissue sections was isolated and used to genotype rs4247357 in CCDC57. Immunostaining was performed on both TMAs using the primary monoclonal antibody against FAS (Transduction Laboratories, Lexington, Ky.) at a 1:100 dilution and hematoxylin as a counterstain. Each core was evaluated for the ratio of stain to counterstain to account for variable cellularity in tissue sections. The average stain/counterstain ratio was determined and compared across myometrium and UL samples and also across samples with the major and minor allele of rs4247357. Error bars and statistical differences were determined by standard error.

mRNA Expression Analysis by Microarray

RNA was isolated from myometrium and 28 UL from 14 women. Gene expression levels were analyzed using the AFFYMETRIX GENECHIP system U133 plus 2.0. DNA was also extracted and used to genotype rs4247357 in CCDC57. Gene expression levels were compared between women with the major and minor allele of rs4247357.

Targeted mRNA Expression Analysis by qPCR

RNA was isolated from myometrium and 20 tumors from 12 women, six with the major allele of rs4247357 and six with the minor allele. cDNA was synthesized and used in qPCR assessments of FASN CCDC57, SLC16A3, DUS1L, (CSNK1D, and NARF. Gene products were normalized to the internal reference gene, GAPDH, and averaged normalized ratios were compared across myometrium and UL samples and across women with the major and minor allele of rs4247357. Error bars and statistical differences were determined by standard error.

### Results

### Linkage Study

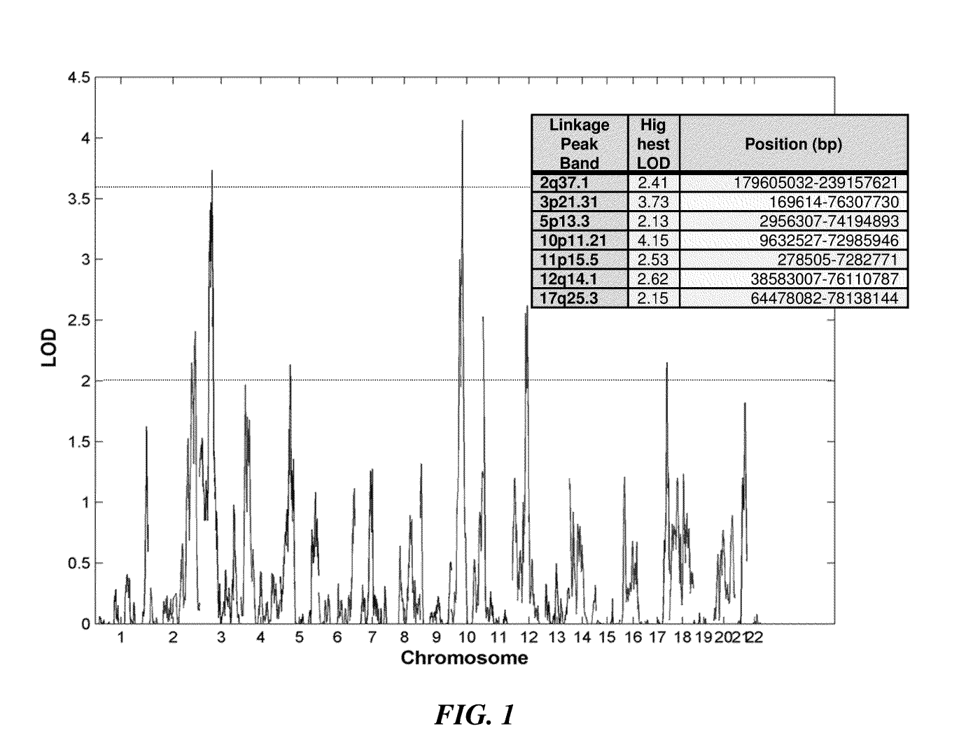

Linkage analysis with the Finding Genes for Fibroids (FGFF) population revealed two peaks with highly significant genome-wide LOD scores (>3.6) and five peaks with suggestive LOD scores (>2.0) (FIG. 1). The highest LOD scores found are at 10p11 (LOD=4.15) and at 3p21 (LOD=3.73). Both linkage regions are around 35 Mb and contain hundreds of genes. Of note, HMGA2 resides within the linkage region at 12q14 with a suggestive LOD score of 2.62 (Supplementary FIG. 1).

### Association Analyses and Meta-Analysis

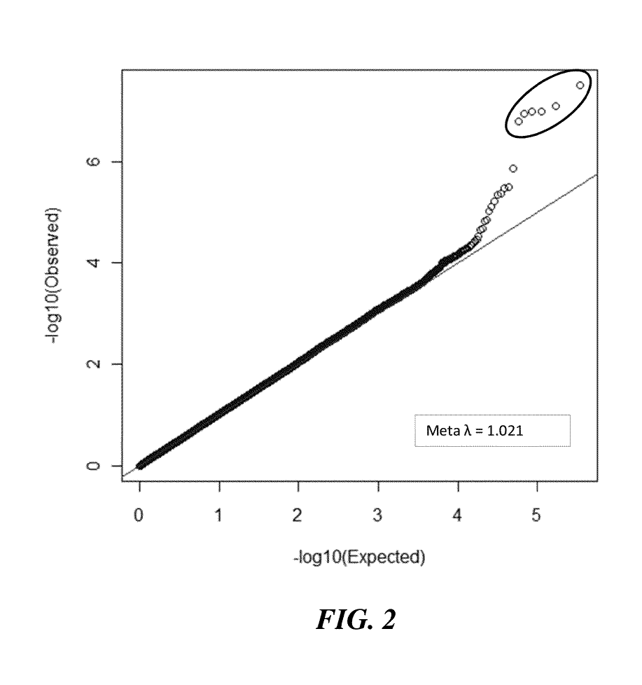

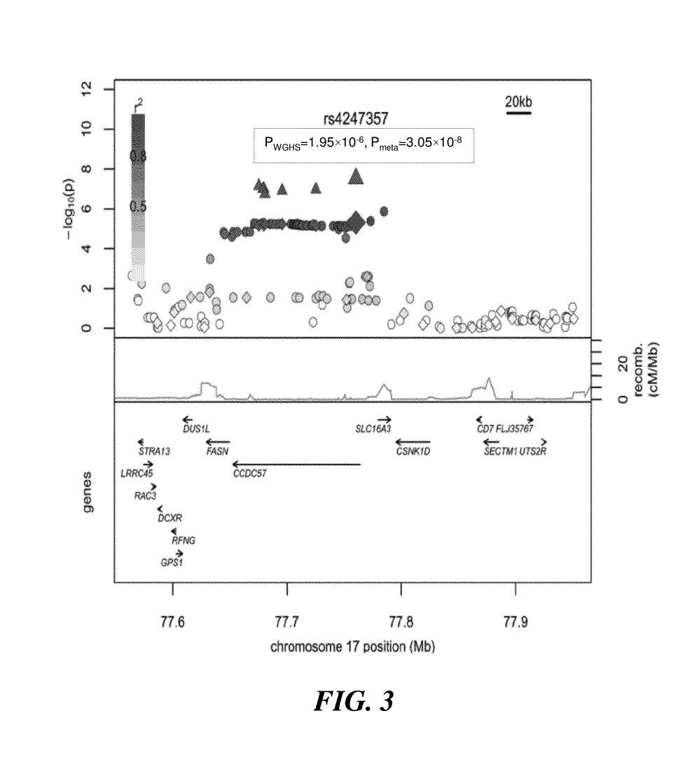

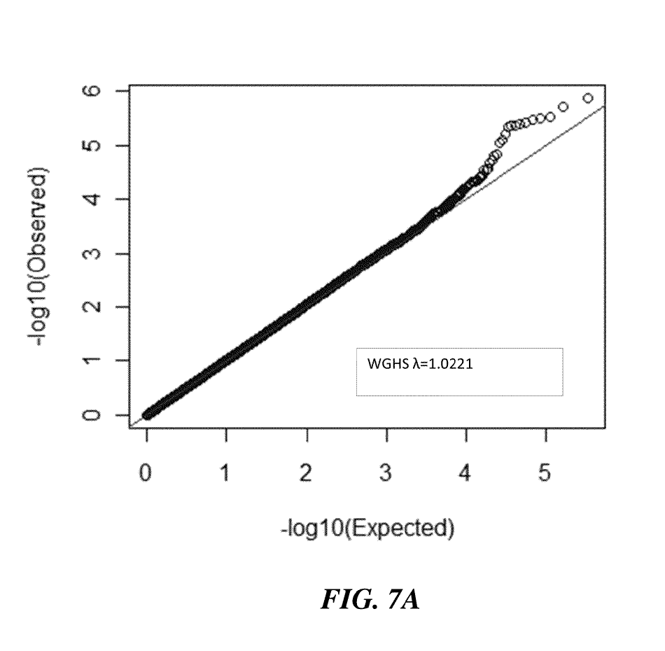

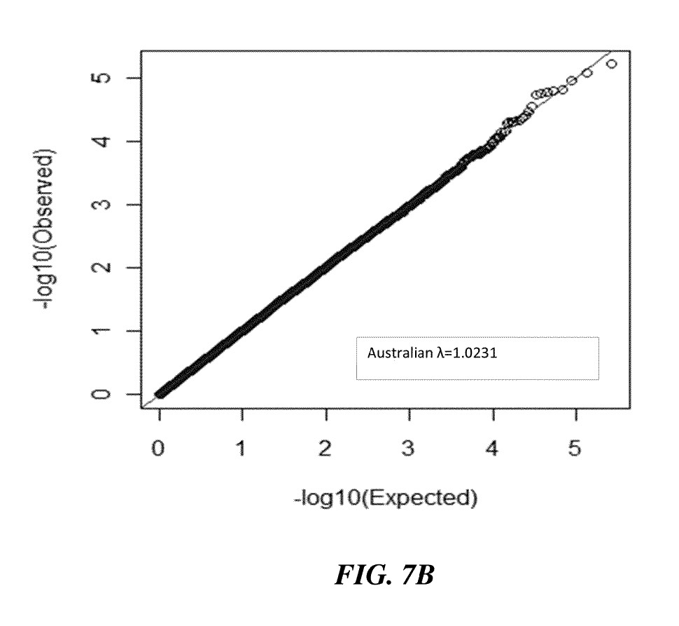

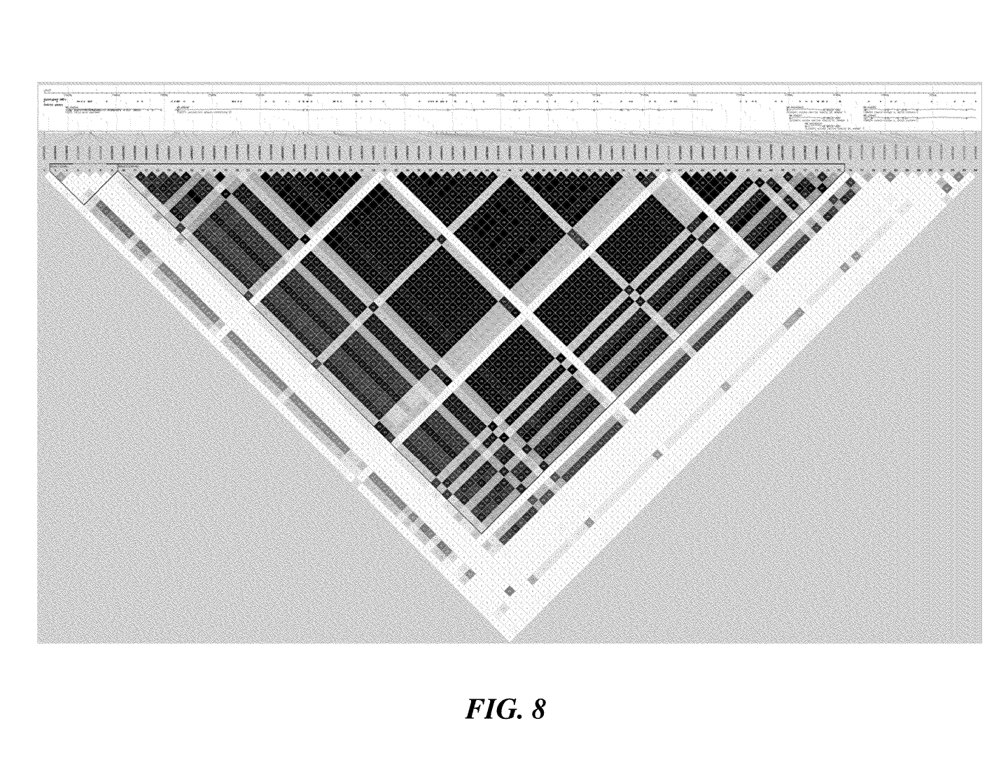

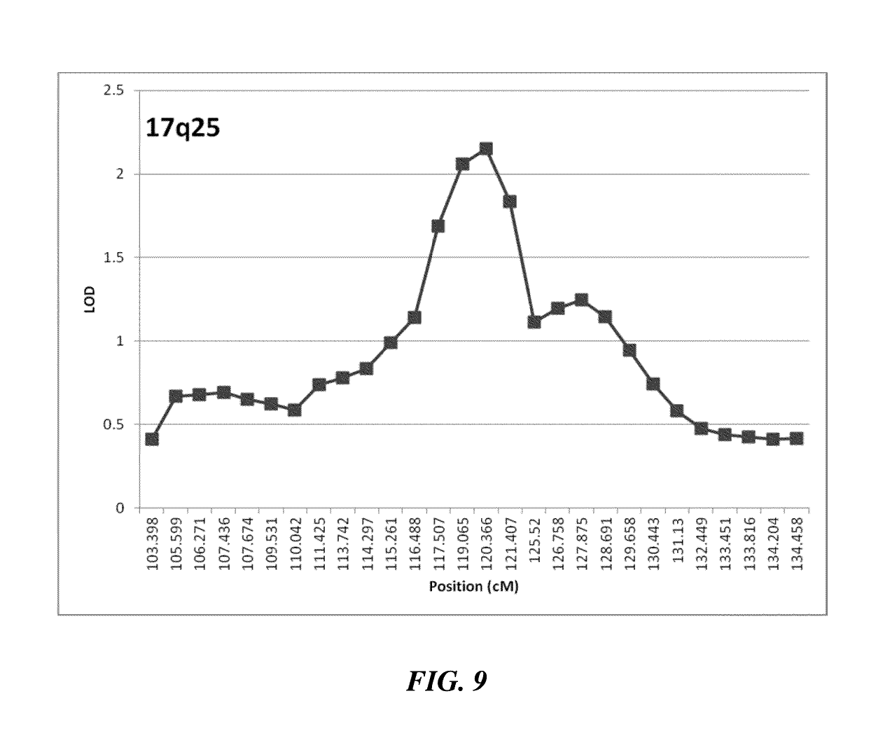

Genome-wide association studies (GWAS) were undertaken with two independent cohorts of white women, the Women's Genome Health Study (WGHS) cohort and an Australian cohort. Analysis of the WGHS cohort revealed 45 SNPs with p-values less than 10⁻⁴ (Table 3). Although none of the p-values from this analysis are considered significant to identify a genome-wide association, the quantile-quantile plot of the results provides evidence that there are more SNPs with small p-values than expected by chance (FIG. 7A). In the Australian analysis, 25 SNPs were identified with p-values less than 10⁻⁴ and the quantile-quantile plot reveals a small increase in low p-values although it is less striking than the WGHS results (Table 4, FIG. 7B). Meta-analysis was performed on the set of 344,655 genotyped SNPs from the WGHS and Australian cohorts using an inverse-variance weighted method in METAL (Meta Analysis Helper for SNP data, World Wide Web site at the University of Michigan, at sph.umich.edu/csg/abecasis/Metal). One SNP, rs4247357, reached genome-wide significance and is considered significantly associated with UL status (Table 1). Five additional SNPs in the same location on chromosome 17 were identified with p-values less than 10-6. The quantile-quantile plot of the meta-analysis results clearly shows that this group of SNPs has lower p-values than would be expected under the null hypothesis (FIG. 2). The candidate SNPs on chromosome 17 are located in a large linkage disequilibrium (LD) block which contains three genes, fatty acid synthase (FASN), coiled-coil domain containing 57 (CCDC57) and solute carrier family 16, member 3 (SLC/6A3) (FIGS. 3 and 8). Interestingly, this LD block lies under the FGFF linkage peak at 17q25 (FIG. 9).

### FAS Protein Levels

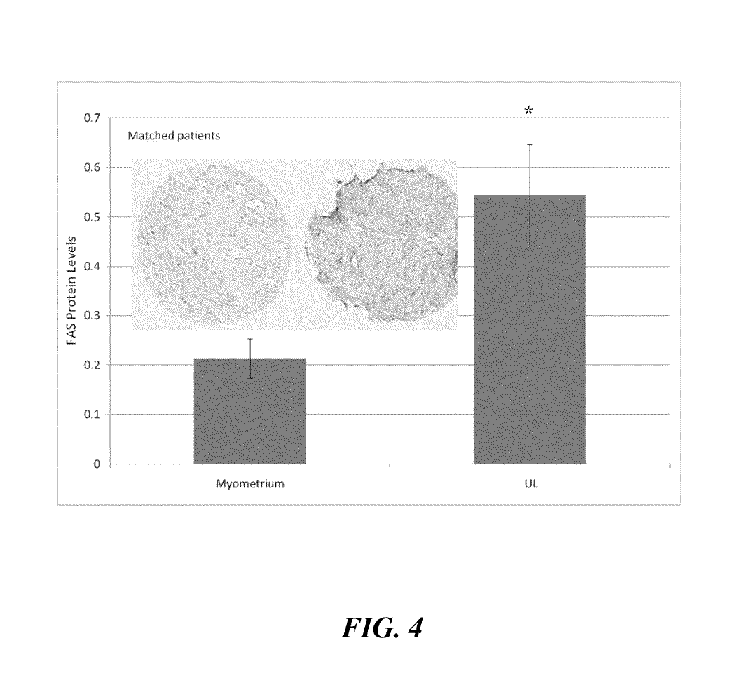

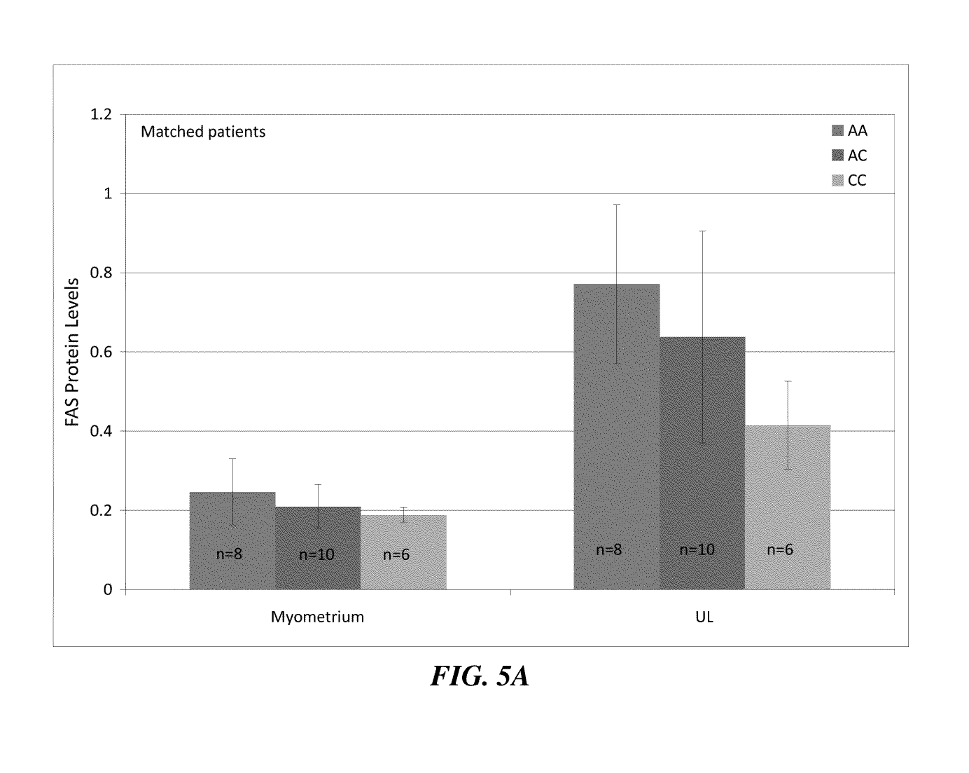

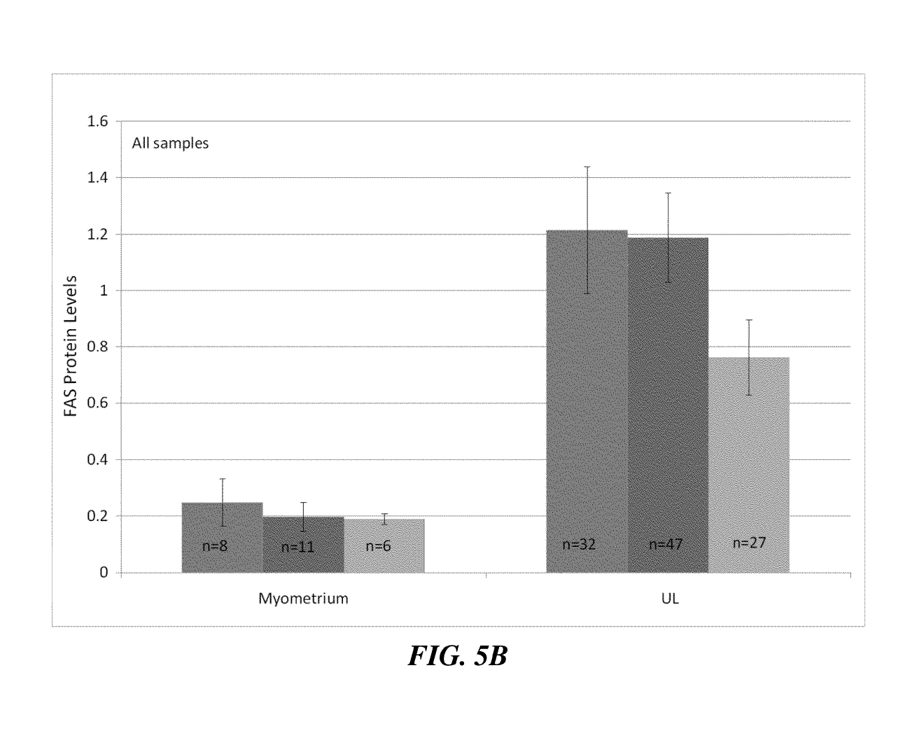

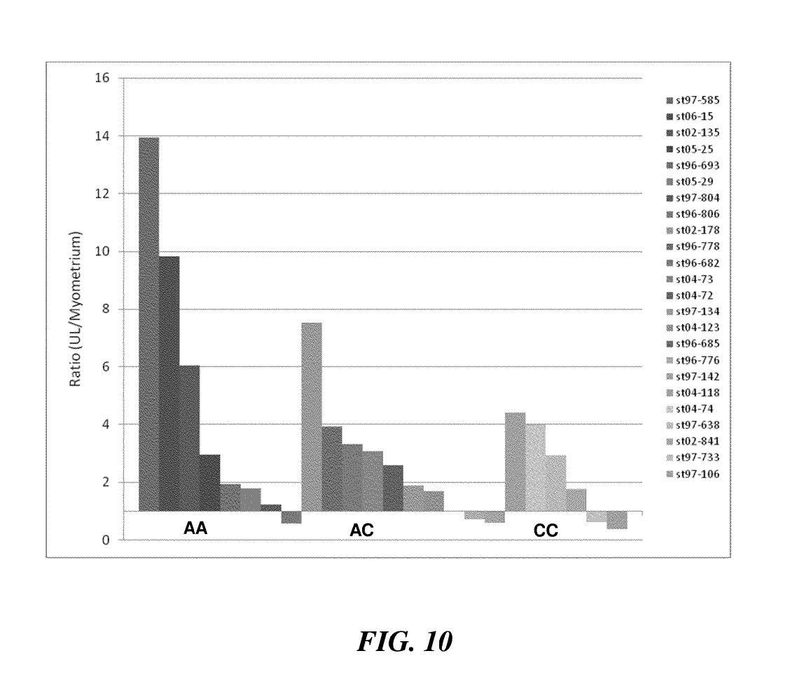

Little is known about CCDC57 or SLC16A3, however, FAS has been associated in several cancers and is often up-regulated in cancer tissue24. In order to investigate this finding in UL, we stained UL and myometrial tissue with a FAS antibody. FAS immunostaining revealed a three-fold increase in FAS levels in UL compared to matched, normal myometrium (FIG. 4). An increase in UL FAS was seen in 25 out of 33 (˜76%) matched samples (FIG. 10). Stratifying matched samples by rs4247357 genotype shows an increase in FAS levels in myometrium and UL with the minor (effect) allele compared to the major allele. Increased expression in myometrium samples with the minor allele (A) is not substantial but the increase in UL with the minor allele (A) is about two-fold (FIG. 5A). When all UL samples are combined, the same pattern emerges but the increase in FAS in UL with the minor allele is about 50% higher than UL with the major allele (FIG. 5B). Interestingly, in the matched analysis, FAS levels in both myometrium and UL samples heterozygous for major and minor alleles was between that of samples homozygous for either the major or minor alleles.

mRNA Expression

Analysis of mRNA expression levels in myometrium and UL by microarray of genes under the linkage peak on chromosome 17 revealed no genes with significant differential expression between women with the major (n=3) and minor alleles (n=6) of rs4247357. FASN, CCDC57 and SLC16A3 were found not to be expressed at a detectable level in the myometrium and UL samples analyzed. However, a more comprehensive analysis of 54,000 probes across the genome revealed several genes significantly upregulated and downregulated in UL with the minor allele compared to UL with the major allele (Table 5).

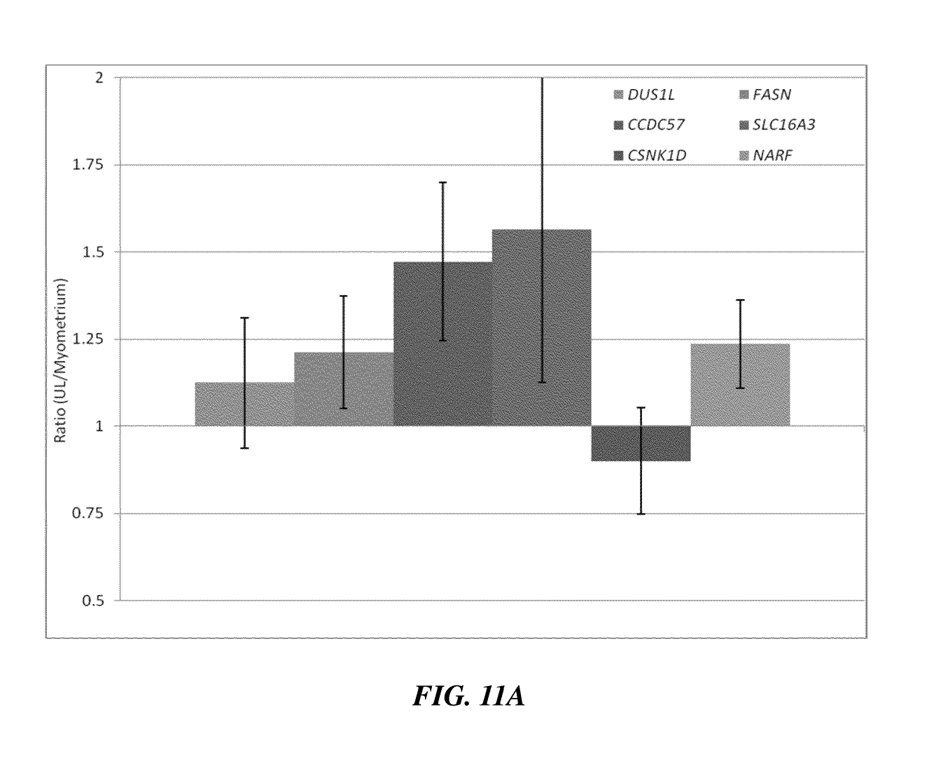

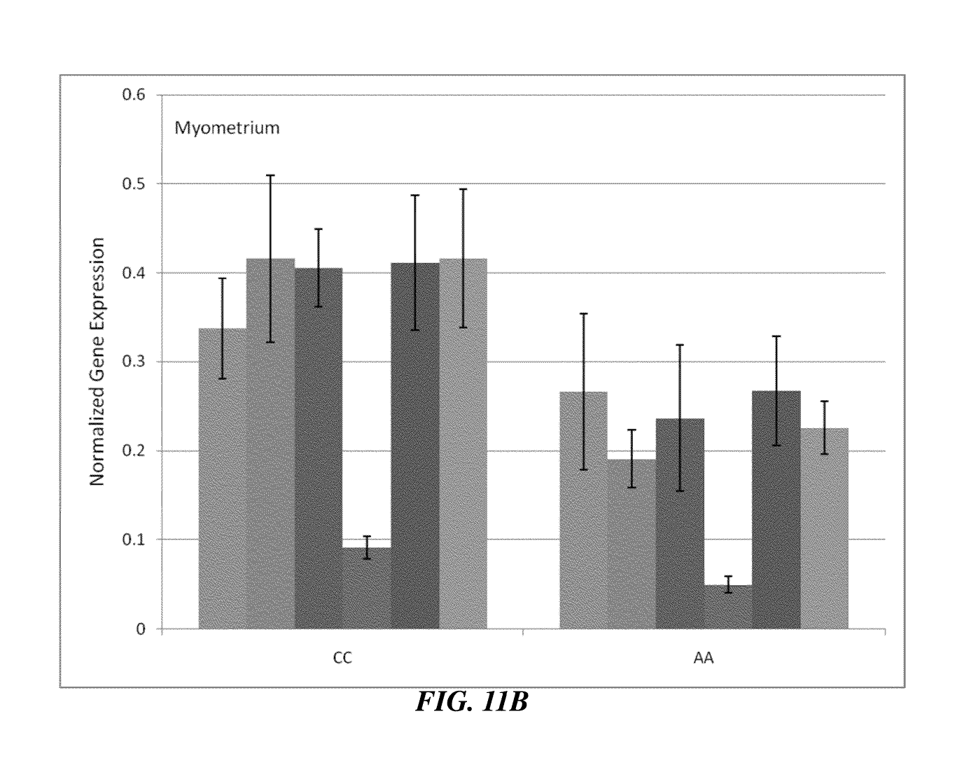

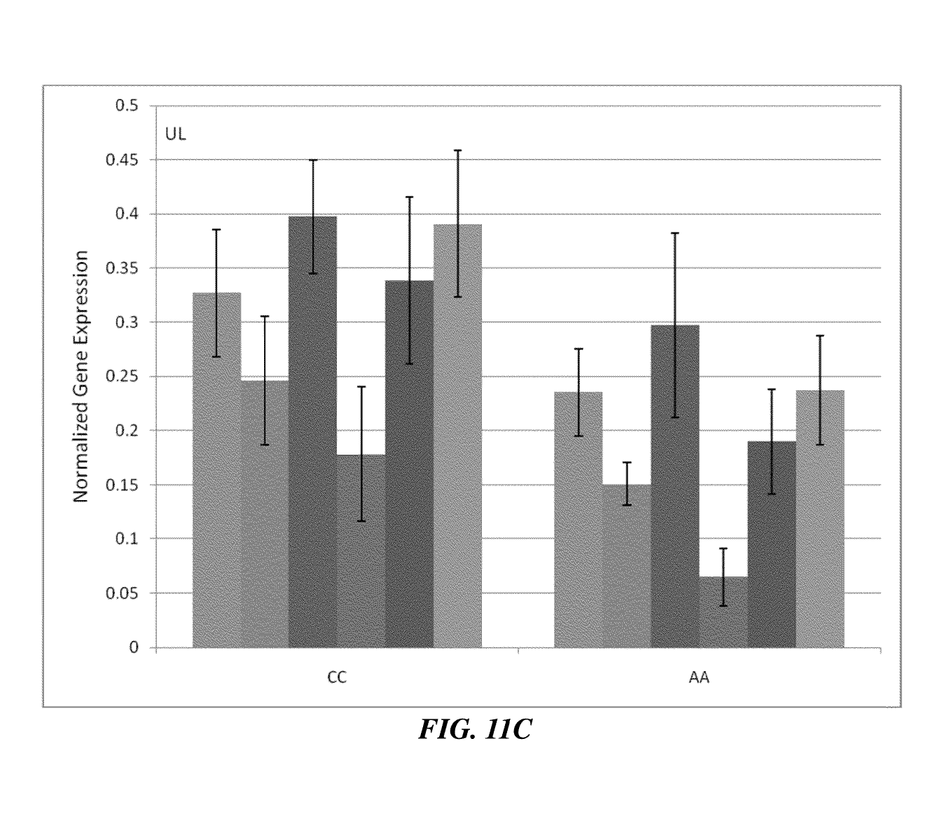

Targeted mRNA expression analysis by qPCR detected no substantial expression differences of matched myometrium and UL samples for FASN. CCDC57, SLC16A3 and three genes located directly nearby but outside of the candidate LD block, DUS1L, CSNK1D and NARF (FIG. 11A). Expression of FASN, CCDC57 and SLC 16A3 is slightly higher in the matched UL samples but expression of DUS1L and NARF is also higher in the matched UL samples and the variation between samples is relatively large. It is unclear whether this expression difference has biological significance; however, it is clear that mRNA expression of FASN between myometrium and matched UL samples is not concordant with FAS protein expression as the same samples were used in both studies. This finding is not surprising as the correlation between mRNA and protein levels can be poor, with some studies finding that only 40% of variation in protein expression can be explained by mRNA expression (25). Also, expression of all six genes was higher in myometrium and UL with the major allele compared to myometrium and UL with the minor allele (FIGS. 11B and 1 IC). Similarly, it is unclear if this difference is of relevance due to the fact that it is relatively small, is observed for every gene analyzed, and the variation between samples is relatively large.

The FGFF linkage study provides evidence for a genetic contribution to UL development as seen by two significant linkage peaks and several other suggestive peaks. Although candidate genes have yet to be identified under five peaks, one gene prominently recognized in UL biology, HMGA2, is located under the peak on 12q14. Translocations involving HMGA2 are frequently found in UL tumors (14). Additionally, a recent study using the FGFF population found a significant association between UL status and a variant in the 5′ UTR of HMGA2 (15). The discovery of several linkage peaks illustrates the genetic heterogeneity of these tumors and is consistent with the hypothesis that a variety of genes and pathways participate in UL development. Additionally, a genome-wide association study in a Japanese cohort found three loci significantly associated with UL diagnosis: 10q24.33, 22q13.1, and 11p15.5 (26). These loci are not associated with UL in our cohorts of white women, and our locus at 17q25.3 was not identified in the Japanese study supporting genetic heterogeneity in UL predisposition between ethnic groups.

Association analyses using the WGHS and Australian cohorts may have identified more precisely the locus underlying the linkage signal on chromosome 17. Six SNPs on chromosome 17 were in the top associated SNPs in the WGHS analysis and proved significantly associated with UL status after meta-analysis of the WGHS and Australian cohorts. These data show that the minor allele frequency of these SNP in chromosome 17 is significantly higher in women with UL. These SNPs are in LD across FASN, CCDC57 and SLC16A3. SLC16A3 is a member of the proton-linked monocarboxylate transporter family that facilitates transport of substrates across the plasma membrane. CCDC57 contains a coiled coil domain and may function by binding DNA. SLC16A3 and CCDC57 are minimally characterized, making it difficult to conjecture about their possible involvement in UL development. Neither of these genes nor proteins has been indicated in disease and our qPCR studies did not reveal any unusual mRNA expression in UL. Conversely, the investigators found a large increase in FAS protein levels in UL compared to matched myometrial tissue and in myometrium and UL from women with the minor (effect) allele (A) compared to women with the major allele (C). FAS protein has been extensively characterized and is the enzyme responsible for die novo fatty acid synthesis. It is most highly expressed in hormone-sensitive cells (27) and has been found to be regulated at both transcriptional and post-transcriptional levels. Sterol regulatory element binding transcription factor 1 (SREBP-1) is the primary transcription factor of FASN and is activated downstream of growth factor and hormone receptors (28). FAS protein is stabilized by USP2a, an isopeptidase that deubiquitinates and prevents protein degradation. USP2a is overexpressed in some prostate tumors with high FAS expression (29) and could explain discordant levels of FASN mRNA and FAS protein levels in our UL samples.

Upregulation of FAS has been discovered in many cancers including prostate, breast, and colon (30-32). In some cancers, upregulation of FAS is correlated with poor prognosis, cancer progression or specific tumor types (33). Inhibitors of FAS lead to growth arrest and apoptosis in cancer cell lines with relatively minor effects in corresponding normal cells (34; 35). Many studies have found a connection between FAS and the PI3K/Akt signaling pathway, one of the most frequently disregulated pathways in human cancers (36; 37). Inhibitors of FAS used in breast cancer animal models and several xenograft models have resulted in delayed development and slowed progression of tumors (38; 39). Knocking down FASN mRNA causes a host of changes in gene expression and protein activity in the cell (40) making it clear from these and many more studies that the role of FAS in neoplasia is much more complicated and probably more important than simply providing fatty acids. Although it remains to be known how the minor allele of the LD block influences fASN and UL development, upregulation of the FAS protein in these UL samples and the overwhelming evidence that FAS is a metabolic oncogene makes it a compelling gene for UL development and warrant targeting the gene as well as the protein with inhibitors for treatment and management of the UL condition in afflicted women.

The references cited herein and throughout the specification are incorporated herein by reference.
1. Cramer, S. F., and Patel, A. (1990). The frequency of uterine leiomyomas. Am J Clin Pathol 94, 435-438.
2. Buttram, V. C., Jr., and Reiter, R. C. (1981). Uterine leiomyomata: etiology, symptomatology, and management. Fertil Steril 36, 433-445.
3. Lepine, L. A., Hillis, S. D., Marchbanks, P. A., Koonin, L. M., Morrow, B., Kieke, B. A., and Wilcox, L. S. (1997). Hysterectomy surveillance—United States, 1980-1993. MMWR CDC Surveill Summ 46, 1-15.
4. Flynn, M., Jamison, M., Datta, S., and Myers, E. (2006). Health care resource use for uterine fibroid tumors in the United States. Am J Obstet Gynecol 195, 955-964.
5. Rein, M. S., Friedman, A. J., Barbieri, R. L., Pavelka, K., Fletcher, J. A., and Morton, C. C. (1991). Cytogenetic abnormalities in uterine leiomyomata. Obstet Gynecol 77, 923-926.
6. Gross, K. L., and Morton, C. C. (2001). Genetics and the development of fibroids. Clin Obstet Gynecol 44, 335-349.
7. Brosens, I., Deprest, J., Dal Cin, P., and Van den Berghe, H. (1998). Clinical significance ofcytogenetic abnormalities in uterine myomas. Fertil Steril 69, 232-235.
8. Christacos, N. C., Quade, B. J., Dal Cin, P., and Morton, C. C. (2006). Uterine leiomyomata with deletions of Ip represent a distinct cytogenetic subgroup associated with unusual histologic features. Genes Chromosomes Cancer 45, 304-312.
9. Huvck, K. L., Panhuysen, C. I., Cuenco, K. T., Zhang, J., Goldhammer, H., Jones, E. S., Somasundaram, P., Lynch, A. M., Harlow, B. L., Lee, H., et al. (2008). The impact of race as a risk factor for symptom severity and age at diagnosis of uterine leiomyomata among affected sisters. Am J Obstet Gynecol 198, 168 e161-169.
10. Marshall, L. M., Spiegelman, D., Barbieri, R. L., Goldman, M. B., Manson, J. E., Colditz, G. A., Willett, W. C., and Hunter, D. J. (1997). Variation in the incidence of uterine leiomyoma among premenopausal women by age and race. Obstet Gynecol 90, 967-973.
11. Vikhlyaeva, E. M., Khodzhaeva, Z. S., and Fantschenko, N. D. (1995). Familial predisposition to uterine leiomyomas. Int J Gynaecol Obstet 51, 127-131.
12. Treloar, S. A., Do, K. A., and Martin, N. G. (1998). Genetic influences on the age at menopause. Lancet 352, 1084-1085.
13. Luoto, R., Kaprio, J., Rutanen, E. M., Taipale, P., Perola, M., and Koskenvuo, M. (2000). Heritability and risk factors of uterine fibroids—the Finnish Twin Cohort study. Maturitas 37, 15-26.
14. Schoenberg Fejzo, M., Ashar, H. R., Krauter, K. S., Powell, W. L., Rein, M. S., Weremowicz, S., Yoon, S. J., Kucherlapati, R. S., Chada, K., and Morton, C. C. (1996). Translocation breakpoints upstream of the HMGIC gene in uterine leiomyomata suggest dysregulation of this gene by a mechanism different from that in lipomas. Genes Chromosomes Cancer 17, 1-6.
15. Hodge, J. C., K, T. C., Huyck, K. L., Somasundaram, P., Panhuysen, C. I., Stewart, E. A., and Morton, C. C. (2009). Uterine leiomyomata and decreased height: a common HMGA2 predisposition allele. Hum Genet 125, 257-263.
16. Purcell, S., Neale, B., Todd-Brown, K., Thomas, L., Ferreira, M. A., Bender, D., Mailer, J., Sklar, P., de Bakker, P. I., Daly, M. J., et al. (2007). PLINK: a tool set for whole-genome association and population-based linkage analyses. Am J Hum Genet 81, 559-575. World Wide Web site at pngu period mgh period Harvard period edu backslash Purcell backslash plink.
17. Ridker, P. M., Chasman, D. I., Zee, R. Y., Parker, A., Rose, L., Cook, N. R., and Buring, J. E. (2008). Rationale, design, and methodology of the Women's Genome Health Study: a genome-wide association study of more than 25,000 initially healthy american women. Clin Chem 54, 249-255.
18. Medland, S. E., Nyholt, D. R., Painter, J. N., McEvoy, B. P., McRae, A. F., Zhu, G., Gordon, S. D., Ferreira, M. A., Wright, M. J., Henders, A. K., et al. (2009). Common variants in the trichohyalin gene are associated with straight hair in Europeans. Am J Hum Genet 85, 750-755.
19. Painter, J. N., Anderson, C. A., Nyholt, D. R., Macgregor, S., Lin, J., Lee, S. H., Lambert, A., Zhao, Z. Z., Roseman, F., Guo, Q., et al. Genome-wide association study identifies a locus at 7p15.2 associated with endometriosis. Nat Genet 43, 51-54.
20. Treloar, S. A., Wicks, J., Nyholt, D. R., Montgomery, G. W., Bahlo, M., Smith, V., Dawson, G., Mackay. I. J., Weeks, D. E., Bennett. S. T., et al. (2005). Genomewide linkage study in 1,176 affected sister pair families identifies a significant susceptibility locus for endometriosis on chromosome 10q26. Am J Hum Genet 77, 365-376.
21. Treloar, S. A., Do, K. A., O'Connor, V. M., O'Connor, D. T., Yeo, M. A., and Martin, N. G. (1999). Predictors of hysterectomy: an Australian study. Am J Obstet (Gynecol 180, 945-954.
22. Kharazmi, E., Fallah, M., and Luoto, R. (2007). Cardiovascular diseases attributable to hysterectomy: a population-based study. Acta Obstet Gynecol Scand 86, 1476-1483.
23. Takeda, T., Sakata, M., Isobe, A., Miyake, A., Nishimoto, F., Ota, Y., Kamiura, S., and Kimura, T. (2008). Relationship between metabolic syndrome and uterine leiomyomas: a case-control study. Gynecol Obstet Invest 66, 14-17.
24. Menendez, J. A., and Lupu, R. (2007). Fatty acid synthase and the lipogenic phenotype in cancer pathogenesis. Nat Rev Cancer 7, 763-777.
25. de Sousa Abreu, R., Penalva, L. O., Marcotte, E. M., and Vogel, C. (2009). Global signatures of protein and mRNA expression levels. Mol Biosyst 5, 1512-1526.
26. Cha, P. C., Takahashi, A., Hosono, N., Low, S. K., Kamatani, N., Kubo, M., and Nakamura, Y. A genome-wide association study identifies three loci associated with susceptibility to uterine fibroids. Nat Genet 43, 447-450.
27. Kusakabe, T., Maeda, M., Hoshi, N., Sugino, T., Watanabe, K., Fukuda, T., and Suzuki, T. (2000). Fatty acid synthase is expressed mainly in adult hormone-sensitive cells or cells with high lipid metabolism and in proliferating fetal cells. J Histochem Cytochem 48, 613-622.
28. Eberle, D., Hegarty, B., Bossard, P., Ferre, P., and Foufelle, F. (2004). SREBP transcription factors: master regulators of lipid homeostasis. Biochimie 86, 839-848.
29. Graner, E., Tang, D., Rossi, S., Baron, A., Migita, T., Weinstein, L. J., Lechpammer, M., Huesken, D., Zimmermann, J., Signoretti, S., et al. (2004). The isopeptidase USP2a regulates the stability of fatty acid synthase in prostate cancer. Cancer Cell 5, 253-261.
30. Alo, P. L., Visca, P., Trombetta, G., Mangoni, A., Lenti, L., Monaco, S., Botti, C., Serpieri, D. E., and Di Tondo, U. (1999). Fatty acid synthase (FAS) predictive strength in poorly differentiated early breast carcinomas. Tumori 85, 35-40.
31. Rossi, S., Graner, E., Febbo, P., Weinstein, L., Bhattacharya, N., Onody, T., Bubley, G., Balk, S., and Loda, M. (2003). Fatty acid synthase expression defines distinct molecular signatures in prostate cancer. Mol Cancer Res 1, 707-715.
32. Ogino, S., Nosho, K., Meyerhardt, J. A., Kirkner, G. J., Chan, A. T., Kawasaki, T., Giovannucci, E. L., Loda, M., and Fuchs, C. S. (2008). Cohort study of fatty acid synthase expression and patient survival in colon cancer. J Clin Oncol 26, 5713-5720.
33. Liu, H., Liu, J. Y., Wu, X., and Zhang, J. T. Biochemistry, molecular biology, and pharmacology of fatty acid synthase, an emerging therapeutic target and diagnosis/prognosis marker. Int J Biochem Mol Biol 1, 69-89.
34. Pizer, E. S., Jackisch, C., Wood, F. D., Pasternack, G. R., Davidson, N. E., and Kuhajda, F. P. (1996). Inhibition of fatty acid synthesis induces programmed cell death in human breast cancer cells. Cancer Res 56, 2745-2747.
35. Pizer, E. S., Chrest, F. J., DiGiuseppe, J. A., and Han, W. F. (1998). Pharmacological inhibitors of mammalian fatty acid synthase suppress DNA replication and induce apoptosis in tumor cell lines. Cancer Res 58, 4611-4615.
36. Van de Sande, T., De Schrijver, E., Heyns, W., Verhoeven, G., and Swinnen, J. V. (2002). Role of the phosphatidylinositol 3′-kinase/PTEN/Akt kinase pathway in the overexpression of fatty acid synthase in LNCaP prostate cancer cells. Cancer Res 62, 642-646.
37. Porstmann, T., Griffiths, B., Chung, Y. L., Delpuech, O., Griffiths, J. R., Downward, J., and Schulze, A. (2005). PKB/Akt induces transcription of enzymes involved in cholesterol and fatty acid biosynthesis via activation of SREBP. Oncogene 24, 6465-6481.
38. Alli, P. M., Pinn, M. L., Jaffee, E. M., McFadden, J. M., and Kuhajda, F. P. (2005). Fatty acid synthase inhibitors are chemopreventive for mammary cancer in neu-N transgenic mice. Oncogene 24, 39-46.
39. Lupu, R., and Menendez, J. A. (2006). Pharmacological inhibitors of Fatty Acid Synthase (FASN)—catalyzed endogenous fatty acid biogenesis: a new family of anti-cancer agents? Curr Pharm Biotechnol 7, 483-493.
40. Knowles, L. M., and Smith, J. W. (2007). Genome-wide changes accompanying knockdown of fatty acid synthase in breast cancer. BMC Genomics 8, 168.
41. Chakravarthy M V, Lodhi I J, Yin L, Malapaka R R, Xu H E, Turk J, Semenkovich C F. (2009). Identification of a physiologically relevant endogenous ligand for PPARalpha in liver. Cell. 138 (3): 476-88
42. Wu M, Singh S B, Wang J, Chung C C, Salituro G, Karanam B V, Lee S H, Powles M, Ellsworth K P, Lassman M E, Miller C, Myers R W, Tota M R, Zhang B B, Li C. (2011). Antidiabetic and antisteatotic effects of the selective fatty acid synthase (FAS) inhibitor platensimycin in mouse models of diabetes. Proc Natl Acad Sci USA. 108 (13): 5378-83.
43. Flavin R., S. Peluso, P. L. Nguyen, and M. Loda, (2010) Fatty acid synthase as a potential therapeutic target in cancer, Future Oncol.; 6(4): 551-562.

Homo sapiens fatty acid synthase (FASN), mRNA
NCBI GENBANK ™ Reference Sequence: NM_004104.4
(SEQ. ID. NO: 1)
ATGGAGGAGGTGGTGATTGCCGGCATGTCCGGGAAGCTGCCAGAGTCGGAGAACTTGCAGGAGTTCTGGG
ACAACCTCATCGGCGGTGTGGACATGGTCACGGACGATGACCGTCGCTGGAAGGCGGGGCTCTACGGCCT
GCCCCGGCGGTCCGGCAAGCTGAAGGACCTGTCTAGGTTTGATGCCTCCTTCTTCGGAGTCCACCCCAAG
CAGGCACACACGATGGACCCTCAGCTGCGGCTGCTGCTGGAAGTCACCTATGAAGCCATCGTGGACGGAG
GCATCAACCCAGATTCACTCCGAGGAACACACACTGGCGTCTGGGTGGGCGTGAGCGGCTCTGAGACCTC
GGAGGCCCTGAGCCGAGACCCCGAGACACTCGTGGGCTACAGCATGGTGGGCTGCCAGCGAGCGATGATG
GCCAACCGGCTCTCCTTCTTCTTCGACTTCAGAGGGCCCAGCATCGCACTGGACACAGCCTGCTCCTCCA
GCCTGATGGCCCTGCAGAACGCCTACCAGGCCATCCACAGCGGGCAGTGCCCTGCCGCCATCGTGGGGGG
CATCAATGTCCTGCTGAAGCCCAACACCTCCGTGCAGTTCTTGAGGCTGGGGATGCTCAGCCCCGAGGGC
ACCTGCAAGGCCTTCGACACAGCGGGGAATGGGTACTGCCGCTCGGAGGGTGTGGTGGCCGTCCTGCTGA
CCAAGAAGTCCCTGGCCCGGCGGGTGTACGCCACCATCCTGAACGCCGGCACCAATACAGATGGCTTCAA
GGAGCAAGGCGTGACCTTCCCCTCAGGGGATATCCAGGAGCAGCTCATCCGCTCGTTGTACCAGTCGGCC
GGAGTGGCCCCTGAGTCATTTGAATACATCGAAGCCCACGGCACAGGCACCAAGGTGGGCGACCCCCAGG
AGCTGAATGGCATCACCCGAGCCCTGTGCGCCACCCGCCAGGAGCCGCTGCTCATCGGCTCCACCAAGTC
CAACATGGGGCACCCGGAGCCAGCCTCGGGGCTGGCAGCCCTGGCCAAGGTGCTGCTGTCCCTGGAGCAC
GGGCTCTGGGCCCCCAACCTGCACTTCCATAGCCCCAACCCTGAGATCCCAGCGCTGTTGGATGGGCGGC
TGCAGGTGGTGGACCAGCCCCTGCCCGTCCGTGGCGGCAACGTGGGCATCAACTCCTTTGGCTTCGGGGG
CTCCAACGTGCACATCATCCTGAGGCCCAACACGCAGCCGCCCCCCGCACCCGCCCCACATGCCACCCTG
CCCCGTCTGCTGCGGGCCAGCGGACGCACCCCTGAGGCCGTGCAGAAGCTGCTGGAGCAGGGCCTCCGGC
ACAGCCAGGACCTGGCTTTCCTGAGCATGCTGAACGACATCGCGGCTGTCCCCGCCACCGCCATGCCCTT
CCGTGGCTACGGTGTGCTGGGTGGTGAGCGCGGTGGCCCAGAGGTGCAGCAGGTGCCCGCTGGCGAGCGC
CCGCTCTGGTTCATCTGCTCTGGGATGGGCACACAGTGGCGCGGGATGGGGCTGAGCCTCATGCGCCTGG
ACCGCTTCCGAGATTCCATCCTACGCTCCGATGAGGCTGTGAAGCCATTCGGCCTGAAGGTGTCACAGCT
GCTGCTGAGCACAGACGAGAGCACCTTTGATGACATCGTCCATTCGTTTGTGAGCCTGACTGCCATCCAG
ATAGGCCTCATAGACCTGCTGAGCTGCATGGGGCTGAGGCCAGATGGCATCGTCGGCCACTCCCTGGGGG
AGGTGGCCTGTGGCTACGCCGACGGCTGCCTGTCCCAGGAGGAGGCCGTCCTCGCTGCCTACTGGAGGGG
ACAGTGCATCAAAGAAGCCCATCTCCCGCCGGGCGCCATGGCAGCCGTGGGCTTGTCCTGGGAGGAGTGT
AAACAGCGCTGCCCCCCGGGCGTGGTGCCCGCCTGCCACAACTCCAAGGACACAGTCACCATCTCGGGAC
CTCAGGCCCCGGTGTTTGAGTTCGTGGAGCAGCTGAGGAAGGAGGGTGTGTTTGCCAAGGAGGTGCGGAC
CGGCGGTATGGCCTTCCACTCCTACTTCATGGAGGCCATCGCACCCCCACTGCTGCAGGAGCTCAAGAAG
GTGATCCGGGAGCCGAAGCCACGTTCAGCCCGCTGGCTCAGCACCTCTATCCCCGAGGCCCAGTGGCACA
GCAGCCTGGCACGCACGTCCTCCGCCGAGTACAATGTCAACAACCTGGTGAGCCCTGTGCTGTTCCAGGA
GGCCCTGTGGCACGTGCCTGAGCACGCGGTGGTGCTGGAGATCGCGCCCCACGCCCTGCTGCAGGCTGTC
CTGAAGCGTGGCCTGAAGCCGAGCTGCACCATCATCCCCCTGATGAAGAAGGATCACAGGGACAACCTGG
AGTTCTTCCTGGCCGGCATCGGCAGGCTGCACCTCTCAGGCATCGACGGGAACCCCAATGCCTTGTTCCC
ACCTGTGGAGTTCCCAGCTCCCCGAGGAACTCCCCTCATCTCCCCACTCATCAAGTGGGACCACAGCCTG
GCCTGGGACGTGCCGGCCGCCGAGGACTTCCCCAACGGTTCAGGTTCCCCCTCAGCCGCCATCTACAACA
TCGACACCAGCTCCGAGTCTCCTGACCACTACCTGGTGGACCACACCCTCGACGGTCGCGTCCTCTTCCC
CGCCACTGGCTACCTGAGCATAGTGTGGAAGACGCTGGCCCGCGCCCTGGGCCTGGGCGTCGAGCAGCTG
CCTGTGGTGTTTGAGGATGTGGTGCTGCACCAGGCCACCATCCTGCCCAAGACTGGGACAGTGTCCCTGG
AGGTACGGCTCCTGGAGGCCTCCCGTGCCTTCGAGGTGTCAGAGAACGGCAACCTGGTAGTGAGTGGGAA
GGTGTACCAGTGGGATGACCCTGACCCCAGGCTCTTCGACCACCCGGAAAGCCCCACCCCCAACCCCACG
GAGCCCCTCTTCCTGGCCCAGGCTGAAGTTTACAAGGAGCTGCGTCTGCGTGGCTACGACTACGGCCCTC
ATTTCCAGGGCATCCTGGAGGCCAGCCTGGAAGGTGACTCGGGGAGGCTGCTGTGGAAGGATAACTGGGT
GAGCTTCATGGACACCATGCTGCAGATGTCCATCCTGGGCTCGGCCAAGCACGGCCTGTACCTGCCCACC
CGTGTCACCGCCATCCACATCGACCCTGCCACCCACAGGCAGAAGCTGTACACACTGCAGGACAAGGCCC
AAGTGGCTGACGTGGTGGTGAGCAGGTGGCTGAGGGTCACAGTGGCCGGAGGCGTCCACATCTCCGGGCT
CCACACTGAGTCGGCCCCGCGGCGGCAGCAGGAGCAGCAGGTGCCCATCCTGGAGAAGTTTTGCTTCACT
CCCCACACGGAGGAGGGGTGCCTGTCTGAGCGCGCTGCCCTGCAGGAGGAGCTGCAACTGTGCAAGGGGC
TGGTGCAGGCACTGCAGACCAAGGTGACCCAGCAGGGGCTGAAGATGGTGGTGCCCGGACTGGATGGGGC
CCAGATCCCCCGGGACCCCTCACAGCAGGAACTGCCCCGGCTGTTGTCGGCTGCCTGCAGGCTTCAGCTC
AACGGGAACCTGCAGCTGGAGCTGGCGCAGGTGCTGGCCCAGGAGAGGCCCAAGCTGCCAGAGGACCCTC
TGCTCAGCGGCCTCCTGGACTCCCCGGCACTCAAGGCCTGCCTGGACACTGCCGTGGAGAACATGCCCAG
CCTGAAGATGAAGGTGGTGGAGGTGCTGGCTGGCCACGGTCACCTGTATTCCCGCATCCCAGGCCTGCTC
AGCCCCCATCCCCTGCTGCAGCTGAGCTACACGGCCACCGACCGCCACCCCCAGGCCCTGGAGGCTGCCC
AGGCCGAGCTGCAGCAGCACGACGTTGCCCAGGGCCAGTGGGATCCCGCAGACCCTGCCCCCAGCGCCCT
GGGCAGCGCCGACCTCCTGGTGTGCAACTGTGCTGTGGCTGCCCTCGGGGACCCGGCCTCAGCTCTCAGC
AACATGGTGGCTGCCCTGAGAGAAGGGGGCTTTCTGCTCCTGCACACACTGCTCCGGGGGCACCCCCTCG
GGGACATCGTGGCCTTCCTCACCTCCACTGAGCCGCAGTATGGCCAGGGCATCCTGAGCCAGGACGCGTG
GGAGAGCCTCTTCTCCAGGGTGTCGCTGCGCCTGGTGGGCCTGAAGAAGTCCTTCTACGGCTCCACGCTC
TTCCTGTGCCGCCGGCCCACCCCGCAGGACAGCCCCATCTTCCTGCCGGTGGACGATACCAGCTTCCGCT
GGGTGGAGTCTCTGAAGGGCATCCTGGCTGACGAAGACTCTTCCCGGCCTGTGTGGCTGAAGGCCATCAA
CTGTGCCACCTCGGGCGTGGTGGGCTTGGTGAACTGTCTCCGCCGAGAGCCCGGCGGGAACCGCCTCCGG
TGTGTGCTGCTCTCCAACCTCAGCAGCACCTCCCACGTCCCGGAGGTGGACCCGGGCTCCGCAGAACTGC
AGAAGGTGTTGCAGGGAGACCTGGTGATGAACGTCTACCGCGACGGGGCCTGGGGGGCTTTCCGCCACTT
CCTGCTGGAGGAGGACAAGCCTGAGGAGCCGACGGCACATGCCTTTGTGAGCACCCTCACCCGGGGGGAC
CTGTCCTCCATCCGCTGGGTCTGCTCCTCGCTGCGCCATGCCCAGCCCACCTGCCCTGGCGCCCAGCTCT
GCACGGTCTACTACGCCTCCCTCAACTTCCGCGACATCATGCTGGCCACTGGCAAGCTGTCCCCTGATGC
CATCCCAGGGAAGTGGACCTCCCAGGACAGCCTGCTAGGTATGGAGTTCTCGGGCCGAGACGCCAGCGGC
AAGCGTGTGATGGGACTGGTGCCTGCCAAGGGCCTGGCCACCTCTGTCCTGCTGTCACCGGACTTCCTCT
GGGATGTGCCTTCCAACTGGACGCTGGAGGAGGCGGCCTCGGTGCCTGTCGTCTACAGCACGGCCTACTA
CGCGCTGGTGGTGCGTGGGCGGGTGCGCCCCGGGGAGACGCTGCTCATCCACTCGGGCTCGGGCGGCGTG
GGCCAGGCCGCCATCGCCATCGCCCTCAGTCTGGGCTGCCGCGTCTTCACCACCGTGGGGTCGGCTGAGA
AGCGGGCGTACCTCCAGGCCAGGTTCCCCCAGCTCGACAGCACCAGCTTCGCCAACTCCCGGGACACATC
CTTCGAGCAGCATGTGCTGTGGCACACGGGCGGGAAGGGCGTTGACCTGGTCTTGAACTCCTTGGCGGAA
GAGAAGCTGCAGGCCAGCGTGAGGTGCTTGGCTACGCACGGTCGCTTCCTGGAAATTGGCAAATTCGACC
TTTCTCAGAACCACCCGCTCGGCATGGCTATCTTCCTGAAGAACGTGACATTCCACGGGGTCCTACTGGA
TGCGTTCTTCAACGAGAGCAGTGCTGACTGGCGGGAGGTGTGGGCGCTTGTGCAGGCCGGCATCCGGGAT
GGGGTGGTACGGCCCCTCAAGTGCACGGTGTTCCATGGGGCCCAGGTGGAGGACGCCTTCCGCTACATGG
CCCAAGGGAAGCACATTGGCAAAGTCGTCGTGCAGGTGCTTGCGGAGGAGCCGGAGGCAGTGCTGAAGGG
GGCCAAACCCAAGCTGATGTCGGCCATCTCCAAGACCTTCTGCCCGGCCCACAAGAGCTACATCATCGCT
GGTGGTCTGGGTGGCTTCGGCCTGGAGTTGGCGCAGTGGCTGATACAGCGTGGGGTGCAGAAGCTCGTGT
TGACTTCTCGCTCCGGGATCCGGACAGGCTACCAGGCCAAGCAGGTCCGCCGGTGGAGGCGCCAGGGCGT
ACAGGTGCAGGTGTCCACCAGCAACATCAGCTCACTGGAGGGGGCCCGGGGCCTCATTGCCGAGGCGGCG
CAGCTTGGGCCCGTGGGCGGCGTCTTCAACCTGGCCGTGGTCTTGAGAGATGGCTTGCTGGAGAACCAGA
CCCCAGAGTTCTTCCAGGACGTCTGCAAGCCCAAGTACAGCGGCACCCTGAACCTGGACAGGGTGACCCG
AGAGGCGTGCCCTGAGCTGGACTACTTTGTGGTCTTCTCCTCTGTGAGCTGCGGGCGTGGCAATGCGGGA
CAGAGCAACTACGGCTTTGCCAATTCCGCCATGGAGCGTATCTGTGAGAAACGCCGGCACGAAGGCCTCC
CAGGCCTGGCCGTGCAGTGGGGCGCCATCGGCGACGTGGGCATTTTGGTGGAGACGATGAGCACCAACGA
CACGATCGTCAGTGGCACGCTGCCCCAGCGCATGGCGTCCTGCCTGGAGGTGCTGGACCTCTTCCTGAAC
CAGCCCCACATGGTCCTGAGCAGCTTTGTGCTGGCTGAGAAGGCTGCGGCCTATAGGGACAGGGACAGCC
AGCGGGACCTGGTGGAGGCCGTGGCACACATCCTGGGCATCCGCGACTTGGCTGCTGTCAACCTGGACAG
CTCACTGGCGGACCTGGGCCTGGACTCGCTCATGAGCGTGGAGGTGCGCCAGACGCTGGAGCGTGAGCTC
AACCTGGTGCTGTCCGTGCGCGAGGTGCGGCAACTCACGCTCCGGAAACTGCAGGAGCTGTCCTCAAAGG
CGGATGAGGCCAGCGAGCTGGCATGCCCCACGCCCAAGGAGGATGGTCTGGCCCAGCAGCAGACTCAGCT
GAACCTGCGCTCCCTGCTGGTGAACCCGGAGGGCCCCACCCTGATGCGGCTCAACTCCGTGCAGAGCTCG
GAGCGGCCCCTGTTCCTGGTGCACCCAATCGAGGGCTCCACCACCGTGTTCCACAGCCTGGCCTCCCGGC
TCAGCATCCCCACCTATGGCCTGCAGTGCACCCGAGCTGCGCCCCTTGACAGCATCCACAGCCTGGCTGC
CTACTACATCGACTGCATCAGGCAGGTGCAGCCCGAGGGCCCCTACCGCGTGGCCGGCTACTCCTACGGG
GCCTGCGTGGCCTTTGAAATGTGCTCCCAGCTGCAGGCCCAGCAGAGCCCAGCCCCCACCCACAACAGCC
TCTTCCTGTTCGACGGCTCGCCCACCTACGTACTGGCCTACACCCAGAGCTACCGGGCAAAGCTGACCCC
AGGCTGTGAGGCTGAGGCTGAGACGGAGGCCATATGCTTCTTCGTGCAGCAGTTCACGGACATGGAGCAC
AACAGGGTGCTGGAGGCGCTGCTGCCGCTGAAGGGCCTAGAGGAGCGTGTGGCAGCCGCCGTGGACCTGA
TCATCAAGAGCCACCAGGGCCTGGACCGCCAGGAGCTGAGCTTTGCGGCCCGGTCCTTCTACTACAAGCT
GCGTGCCGCTGAGCAGTACACACCCAAGGCCAAGTACCATGGCAACGTGATGCTACTGCGCGCCAAGACG
GGTGGCGCCTACGGCGAGGACCTGGGCGCGGACTACAACCTCTCCCAGGTATGCGACGGGAAAGTATCCG
TCCACGTCATCGAGGGTGACCACCGCACGCTGCTGGAGGGCAGCGGCCTGGAGTCCATCATCAGCATCAT
CCACAGCTCCCTGGCTGAGCCACGCGTGAGCGTGCGGGAGGGCTAG
(SEQ. ID. NO: 2)
CCCUGAGAUCCCAGCGCUGUU
(SEQ. ID. NO: 3)
UGGAGCGUAUCUGUGAGAA
(SEQ. ID. NO: 4)
CCAUGGAGCGUAUCUGUGA
(SEQ. ID. NO: 5)
UGACAUCGUCCAUUCGUUU
(SEQ. ID. NO: 6)
GACGAGAGCACCUUUGAUG
(SEQ. ID. NO: 7)
GAGCGUAUCUGUGAGAAAC

TABLE 1
Top SNP from meta-analysis results of WGHS and Australian
cohorts (P < 5 × 10⁻⁸)

| | Locus Position | | | | |
|---|---|---|---|---|---|
| SNP | (gene) | Cohort | MAF | OR (95% CI) | P-value |
| rs4247357 | 17q25.3 | WGHS | 0.467 | 1.307 (1.170-1.459) | 1.95E−06 |
| | 77760277 | Australian | 0.432 | 1.282 (1.079-1.523) | 4.49E−03 |
| | (CCDC57) | Meta-analysis | | 1.299 (1.184-1.426) | 3.05E−08 |

TABLE 2
Demographics of the FGFF, WGHS and Australian cohorts

| | | WGHS | WGHS | WGHS | | Australian | Australian | Australian | | FGFF |
|---|---|---|---|---|---|---|---|---|---|---|
| | | all | cases | controls | | all | cases | controls | | sisters |
| N | | (5233) | (746) | (4487) | P-value | (1094) | (484) | (610) | P-value | (522) |
| Age in | Yrs | 52.9 | 52.3 | 52.5 | 0.0038 | 41.0 | 44.0 | 39.0 | 0.0001 | 47.0 |
| years | Median | (48.9-59.0) | (48.8-56.9) | (48.7-58.0) | | (34.0-50.0) | (37.0-51.0) | (34.0-48.0) | | (41.0-53.0) |
| (yrs) | (IQR) | | | | | | | | | |
| BMI | kg/m\hat{ }2 | 24.8 | 25.0 | 24.2 | 0.1595 | 23.6 | 24.8 | 23.4 | 0.0019 | 24.1 |
| | Median | (22.3-28.3) | (22.6-28.2) | (22.0-27.4) | | (21.5-27.1) | (22.3-28.3)* | (21.3-26.5) | | (22.0-27.4)** |
| | (IQR) | | | | | | | | | |
| Height | Inches | 65.0 | 64.0 | 65.0 | 0.0528 | 63.8 | 63.4 | 63.8 | 0.0195 | 65.0 |
| | Median | (63.0-66.0) | (63.0-66.0) | (63.0-66.0) | | (61.8-65.4) | (61.8-65.4)* | (61.8-65.7) | | (63.0-66.75) |
| | (IQR) | | | | | | | | | |
| Age at | Yr | 12.0 | 12.0 | 12.0 | 2.82E−05 | 13.0 | 13.0 | 13.0 | 0.0069 | 12.0 |
| menarche | Median | (12.0-13.0) | (11.0-13.0) | (12.0-13.0) | | (12.0-14.0) | (12.0-14.0) | (12.0-14.0) | | (12.0-13.0) |
| | (IQR) | | | | | | | | | |
| Hyster- | Hyster- | | | | | 312 | 263 | 49 | 3.77E-28 | 165 |
| ectomy | ectomy | | | | | (28.5%) | (56.1%) | (8.3%) | | (30.1%) |
| history | N (%) | | | | | | | | | |

*BMI and height were only available for 175 Australian cases.
**BMI is only available for 491 FGFF sisters.

TABLE 3
WGHS genome-wide association analysis results (P < 10⁻⁴)

| CHR | SNP | Position (bp) | F_A | F_U | A2 | CHISQ | P-value |
|---|---|---|---|---|---|---|---|
| 1 | rs2268179 | 22287371 | 0.2091 | 0.1588 | C | 23.37 | 1.34E−06 |
| 17 | rs4247357 | 77760277 | 0.467 | 0.4014 | C | 22.64 | 1.95E−06 |
| 17 | rs6502057 | 77675348 | 0.4628 | 0.3977 | G | 21.78 | 3.06E−06 |
| 15 | rs2903332 | 78340261 | 0.3124 | 0.2542 | G | 21.75 | 3.10E−06 |
| 17 | rs11077969 | 77678921 | 0.4655 | 0.4017 | G | 21.31 | 3.91E−06 |
| 17 | rs7502078 | 77725167 | 0.4678 | 0.4044 | C | 21.24 | 4.04E−06 |
| 17 | rs7213172 | 77678991 | 0.4657 | 0.4023 | A | 21.16 | 4.23E−06 |
| 17 | rs8080423 | 77695567 | 0.4672 | 0.4039 | A | 21.1 | 4.36E−06 |
| 17 | rs7406163 | 77679677 | 0.4772 | 0.4128 | A | 21.06 | 4.46E−06 |
| 17 | ts7221544 | 77680625 | 0.4664 | 0.4042 | A | 20.41 | 6.26E−06 |
| 9 | rs11139665 | 84451020 | 0.05295 | 0.03042 | A | 19.98 | 7.84E−06 |
| 22 | rs732110 | 26345321 | 0.1166 | 0.08168 | A | 19.73 | 8.90E−06 |
| 5 | rs30523 | 132138941 | 0.1516 | 0.1125 | G | 18.71 | 1.52E−05 |
| 1 | rs6693503 | 19859880 | 0.4135 | 0.3555 | G | 18.61 | 1.61E−05 |
| 14 | rs1951054 | 25147247 | 0.3004 | 0.3575 | A | 18.33 | 1.86E−05 |
| 1 | rs2012235 | 68685342 | 0.2332 | 0.2866 | A | 18.11 | 2.09E−05 |
| 4 | rs2333255 | 176819759 | 0.4289 | 0.4874 | C | 17.55 | 2.80E−05 |
| 1 | rs12563321 | 26539088 | 0.2554 | 0.2074 | G | 17.5 | 2.87E−05 |
| 1 | rs9729637 | 5468038 | 0.09182 | 0.06265 | C | 17.45 | 2.95E−05 |
| 14 | rs1257670 | 98562725 | 0.3394 | 0.2867 | A | 17.06 | 3.63E−05 |
| 1 | rs12117956 | 27370168 | 0.305 | 0.3598 | A | 16.89 | 3.97E−05 |
| 9 | rs7855598 | 137454075 | 0.2262 | 0.1813 | A | 16.84 | 4.06E−05 |
| 1 | rs4846689 | 219444057 | 0.1156 | 0.0832 | A | 16.71 | 4.36E−05 |
| 6 | rs6903101 | 138567539 | 0.04625 | 0.02687 | A | 16.69 | 4.39E−05 |
| 17 | rs917538 | 14975565 | 0.4538 | 0.3978 | C | 16.6 | 4.61E−05 |
| 1 | rs6687674 | 27409687 | 0.362 | 0.4182 | G | 16.58 | 4.67E−05 |
| 14 | rs2284230 | 77085649 | 0.175 | 0.2217 | G | 16.5 | 4.88E−05 |
| 10 | rs3824700 | 26395911 | 0.4953 | 0.4388 | A | 16.48 | 4.92E−05 |
| 11 | rs7945105 | 131162979 | 0.3653 | 0.4212 | G | 16.48 | 4.93E−05 |
| 10 | rs1521032 | 26393597 | 0.4946 | 0.4385 | A | 16.3 | 5.41E−05 |
| 1 | rs10902742 | 26548843 | 0.2372 | 0.1923 | C | 16.13 | 5.91E−05 |
| 10 | rs11593128 | 26392957 | 0.4946 | 0.4385 | A | 16.09 | 6.04E−05 |
| 10 | rs3936497 | 28619974 | 0.1034 | 0.0734 | A | 16 | 6.34E−05 |
| 1 | rs1454356 | 189400411 | 0.3495 | 0.4044 | A | 15.97 | 6.42E−05 |
| 9 | rs1556047 | 112083819 | 0.5121 | 0.4565 | A | 15.86 | 6.83E−05 |
| 2 | rs12692335 | 7524145 | 0.2483 | 0.203 | G | 15.84 | 6.90E−05 |
| 8 | rs10955841 | 119015721 | 0.3718 | 0.3198 | A | 15.67 | 7.52E−05 |
| 1 | rs9786944 | 5471098 | 0.08914 | 0.06186 | G | 15.5 | 8.24E−05 |
| 6 | rs6926282 | 112968480 | 0.128 | 0.09509 | G | 15.43 | 8.55E−05 |
| 21 | rs13049184 | 21818485 | 0.4448 | 0.4998 | G | 15.4 | 8.70E−05 |
| 12 | rs12579612 | 93189923 | 0.2426 | 0.1984 | A | 15.39 | 8.75E−05 |
| 10 | rs1521033 | 26422356 | 0.494 | 0.4395 | G | 15.36 | 8.89E−05 |
| 6 | rs627240 | 81042603 | 0.3834 | 0.4387 | C | 15.33 | 9.04E−05 |
| 15 | rs11636483 | 96937674 | 0.2507 | 0.206 | A | 15.25 | 9.44E−05 |
| 16 | rs16953111 | 78954425 | 0.09987 | 0.07114 | G | 15.17 | 9.83E−05 |

TABLE 4
Australian genome-wide association analysis results (P < 10⁻⁴)

| CHR | SNP | Position (bp) | F_A | F_U | A2 | CHISQ | P-value |
|---|---|---|---|---|---|---|---|
| 8 | rs10504743 | 82951580 | 0.04752 | 0.09918 | G | 20.43 | 6.18E−06 |
| 1 | rs10779614 | 212656537 | 0.1777 | 0.2574 | G | 19.84 | 8.43E−06 |
| 1 | rs6703314 | 104976389 | 0.3244 | 0.2397 | A | 19.29 | 1.13E−05 |
| 12 | rs6539579 | 80696724 | 0.2562 | 0.3418 | A | 18.7 | 1.53E−05 |
| 3 | rs4128782 | 89320422 | 0.468 | 0.5607 | G | 18.57 | 1.64E−05 |
| 7 | rs1829993 | 77698210 | 0.469 | 0.5615 | G | 18.49 | 1.71E−05 |
| 5 | rs10515600 | 147316068 | 0.3089 | 0.3975 | C | 18.45 | 1.74E−05 |
| 17 | rs11079098 | 49220927 | 0.3295 | 0.4189 | A | 18.28 | 1.90E−05 |
| 2 | rs750132 | 218548607 | 0.1508 | 0.09262 | G | 17.52 | 2.84E−05 |
| 17 | rs3785655 | 14189892 | 0.2996 | 0.3844 | T | 17.15 | 3.46E−05 |
| 6 | rs9389508 | 137842904 | 0.1405 | 0.08525 | C | 16.87 | 4.00E−05 |
| 18 | rs4542757 | 48452722 | 0.3068 | 0.391 | T | 16.72 | 4.32E−05 |
| 7 | rs6967325 | 77678568 | 0.407 | 0.323 | A | 16.56 | 4.72E−05 |
| 10 | rs2082988 | 128254551 | 0.3409 | 0.4262 | G | 16.54 | 4.77E−05 |
| 20 | rs6116201 | 4033758 | 0.1498 | 0.218 | A | 16.47 | 4.93E−05 |
| 20 | rs10485664 | 38064192 | 0.2293 | 0.1607 | A | 16.48 | 4.93E−05 |
| 17 | rs1859906 | 14191413 | 0.3688 | 0.4549 | G | 16.47 | 4.95E−05 |
| 9 | rs11144978 | 78415148 | 0.3089 | 0.232 | A | 16.36 | 5.25E−05 |
| 1 | rs1808973 | 112289357 | 0.4122 | 0.4975 | T | 15.83 | 6.93E−05 |
| 7 | rs798332 | 77746864 | 0.3946 | 0.3131 | T | 15.78 | 7.11E−05 |
| 3 | rs7429534 | 89273999 | 0.3564 | 0.4402 | C | 15.74 | 7.28E−05 |
| 6 | rs6570048 | 136240287 | 0.4287 | 0.5131 | T | 15.41 | 8.64E−05 |
| 1 | rs3006009 | 242697070 | 0.3533 | 0.4361 | T | 15.41 | 8.65E−05 |
| 1 | rs11120315 | 212653320 | 0.1126 | 0.1721 | C | 15.36 | 8.90E−05 |
| 12 | rs12228394 | 19482075 | 0.01756 | 0.04836 | C | 15.27 | 9.32E−05 |

TABLE 5
mRNA of genes found to be upregulated or downregulated
in UL with the major allele of rs4247357 compared
to UL with the minor allele.

| | | Fold | | |
|---|---|---|---|---|
| Gene | Location | Change | Direction | P-value |
| MMSI9 | 10q24.1 | 1.558 | up | 7.45E−07 |
| SH3BP4 | 2q37.2 | 1.804 | down | 8.42E−07 |
| TUBB2A | 6p25.2 | 2.658 | down | 1.86E−06 |
| NLN | 5q12.3 | 1.617 | down | 2.19E−06 |
| FAM126A/DRCTNNBIA | 7p15.3 | 1.699 | down | 6.49E−06 |
| STX7 | 6q23.2 | 2.014 | down | 6.88E−06 |
| PDS5B/KIAA0979 | 13q13.1 | 1.753 | down | 7.25E−06 |
| CR613961 | 1p36.11 | 2.655 | down | 7.96E−06 |
| CR749816 | 1p21.2 | 1.817 | down | 8.49E−06 |
| PMEPA1 | 20q13.31 | 2.284 | down | 1.16E−05 |
| MAP3K7IP3 | Xp21.2 | 2.581 | down | 1.17E−05 |
| HOOK3 | 8p11.21 | 1.650 | down | 1.46E−05 |
| C12orf47 | 12q24.12 | 1.728 | up | 1.47E−05 |
| ANKRD27 | 19q13.11 | 1.870 | down | 1.55E−05 |
| RAB6C | 2q21.1 | 1.407 | down | 1.71E−05 |
| C6orf162 | 6q15 | 1.585 | up | 1.82E−05 |
| PICALM | 11q14.2 | 1.884 | down | 1.83E−05 |
| c3orf19 | 3p25.1 | 1.310 | up | 2.07E−05 |
| ZSIG13/FZD4 | 11q14.2 | 2.527 | down | 2.15E−05 |
| P1AS1 | 15q23 | 1.685 | up | 2.36E−05 |
| FADS1 | 11q12.2 | 1.948 | down | 2.38E−05 |
| PHF21A/KIAA1696 | 11p11.2 | 1.343 | up | 2.45E−05 |
| RBM15 | 1p13.3 | 1.810 | down | 2.65E−05 |
| DIS3 | 13q22.1 | 1.565 | down | 2.78E−05 |

What is claimed:

1. A method of treatment of uterine leiomyomata (UL) in a subject in need thereof comprising administering a composition comprising an inhibitor of a fatty acid synthase (FAS) and a pharmaceutical acceptable carrier.

2. The method of claim 1, wherein the composition comprising an inhibitor of FAS inhibits FAS activity.

3. The method of claim 1, wherein the inhibitor of FAS activity is selected from the group consisting of a small molecule inhibitor, a polyphenol and a dietary compound.

4. The method of claim 3, wherein the inhibitor of FAS activity is selected from the group consisting of (S)—((S)-1-((2S,3S)-3-hexyl-4-oxooxetan-2-yl)tridecan-2-yl)2-formamido-4-methylpentanoate, 3-Carboxy-4-octyl-2-methylencbutyrolactone, trans-4-Carboxy-5-octyl-3-methylenebutyrolactone (C75), cerulenin, C93 (FAS93), FAS31, C247, GSK837149A, platensimycin, 3-aryl-4-hydroxyquinolin-2(1H)-one scaffold, and bisamide scaffold.

5. The method of claim 3, wherein the inhibitor of FAS activity is selected from the group consisting of epigallocatechin, luteolin, taxifolin, kaempferol, quercetin and apigenin.

6. The method of claim 3, wherein the inhibitor of FAS activity is selected from the group consisting of catechin, soy protein and monounsaturated fatty acid oleic acid (18:1 n-9).

7. The method of claim 3, wherein the inhibitor of FAS activity is selected from the group consisting of polyhydroxylated compounds, cerulenin compounds, and spirocyclic piperidines.

8. The method of claim 1, wherein the composition comprising an inhibitor of FAS inhibits FAS expression.

9. The method of claim 8, wherein the inhibitor of FAS expression is selected from a small molecule and a nucleic acid.

10. The method of claim 8, wherein the inhibitor of FAS expression is a FAS specific RNA interference agent, or a vector encoding a FAS specific RNA interference agent.

11. The method of claim 10, wherein said RNA interference agent is a double stranded RNA (dsRNA).

12. The method of claim 8, wherein said RNA interference agent comprises one or more of the nucleotide sequences of SEQ. ID. NOS: 2-7.

13. The method of claim 1, wherein the composition is formulated for administration by injection, infusion, instillation, vaginal suppository, cervical suppository, urethral suppository, percutaneous implantation or ingestion.

14. The method of claim 1, wherein the subject has been diagnosed with uterine leiomyomata.

15. The method of claim 1, wherein the subject has the minor SNP allele, adenine (A) at rs4247357 (SEQ ID NO: 9) at chromosome 17.

16. The method of claim 15, wherein the subject is homozygous or heterozygous for the minor (A) SNP allele at rs4247357 (SEQ ID NO: 9) at chromosome 17.

17. A method of treatment of uterine leiomyomata (UL) in a female subject comprising:
a. determining the single nucleotide polymorphism (SNP) allelic genotype at rs4247357 (SEQ ID NO: 9) at chromosome 17 in a genetic sample obtained from a female subject, wherein a homozygosity (AA) or heterozygosity (AC) for the minor SNP allele indicates an increased likelihood of developing UL;
b. administering a composition comprising an inhibitor of a fatty acid synthase (FAS) and a pharmaceutical acceptable carrier to the subject.

18. The method of claim 17, wherein the genetic sample from a female subject is a tissue sample comprising deoxyribonucleic acid (DNA).

19. The method of claim 18, wherein the genetic sample from a female subject is selected from the group consisting of a blood sample, a saliva sample, a skin sample, a hair bulb and an epithelial sample.

20. The method of claim 1, wherein the female subject is selected from the group consisting of having reached puberty, has not entered perimenopause or menopause, has entered perimenopause or menopause, and has not reached menarche.

21. The method of claim 1, wherein the female subject is on hormone replacement therapy.

22. The method of claim 1, wherein the female subject is between the ages of 7-70.

23. The method of claim 1, wherein the female subject has at least one first and/or second degree relative who have had UL.
